(19) United States
(12) Patent Application Publication
(10) Pub. No.: US 2012/0003296 A1
(43) Pub. Date: Jan. 5, 2012

(54) NEW METHODS OF TREATING DRY EYE SYNDROME

(76) Inventors: Totada R. Shantha, McDonough, GA (US); Erica Maya Shantha, McDonough, GA (US); Jessica Gowramma Shantha, McDonough, GA (US)

(21) Appl. No.: 12/828,292

(22) Filed: Jul. 1, 2010

(51) Int. Cl. A61K 9/127; A61K 38/30; A61P 27/02; A61K 38/21; A61K 36/906; A61P 27/04; A61K 38/28; A61K 39/395

(52) U.S. Cl. 424/450; 514/5.9; 514/8.6; 424/141.1; 424/85.7; 424/133.1; 424/756

(57) ABSTRACT

The invention relates to a method of insulin eye drops for treating dry eye syndrome due to any and all etiological factors (Keratoconjunctivitis sicca), including Sjogren's syndrome, Meibomian gland dysfunction (MGD) and other glandular malfunction in the eye lids, lacrimal glands, cornea, conjunctiva, and exposed scleral surface of the eye. It is treated with Insulin and/or IGF-I with or without known anti-dry eye syndrome therapeutic, pharmaceutical, biochemical and biological agents or compounds.

### FIELD OF THE INVENTION

This invention relates to the treatment of dry eyes syndrome (dry eye diseases), and more particularly the treatment of human tear producing glandular function disorders involving oil (meibomian and glands of Zeis), mucus (Goblet cells and ocular surface epithelium via its loose attachments to the glycocalyx of the microplicae of the epithelium), watery tear secreting (lacrimal) glands of the eye lids, cornea and sclera leading to dryness of the eyes and other vision afflictions.

### BACKGROUND OF THE INVENTION

Dry eye syndrome (DES) is a multi factorial disease of the tears, their production resulting in ocular surface pathology associated with symptoms of discomfort, visual disturbance, and tear film instability with potential damage to the ocular surface (exposed eye ball, sclera, conjunctival sac, cornea). Dry eye is associated with increased osmolality of the tear film and inflammation of the ocular surface due to improper secretion production of various secretory glands in the eye lids including lacrimal glands.

Keratoconjunctivitis sicca (KCS) is another name given to dry eye syndrome of the ocular surface disorder. KCS is subdivided into Sjogren's syndrome (SS) associated KCS and non-SS associated KCS. Patients with aqueous tear deficiency have SS if they have associated xerostomia and/or connective tissue disease with evidence of systemic autoimmune diseases. Secondary SS is defined as KCS associated with a diagnosable connective tissue disease such as rheumatoid arthritis, SLE, and systemic scleroderma.

Non Sjogren's syndrome KCS is common in postmenopausal woman, pregnant woman, in women who are taking oral contraceptives, or in women who are on hormone replacement therapy with estrogens. The common denominator here is a decrease in androgens, either from reduced ovarian function in the postmenopausal female or from increased levels of the sex hormone binding globulin in pregnancy and birth control pill use. Androgens are believed to be trophic for the lacrimal and meibomian glands. They also exert potent anti-inflammatory activity through the production of transforming growth factor beta (TGF-beta), suppressing lymphocytic infiltration. Lipocalins (pre albumin), present in the mucous layer, produced by the lacrimal glands lowers the surface tension of normal tears providing the stability to the tear film and also explains the increase in surface tension seen in dry eye syndromes characterized by lacrimal gland deficiency. Lipocalin deficiency can lead to the precipitation in the tear film, forming the characteristic mucous strands seen in patients with dry eye syndrome symptoms.

The glycocalyx of the corneal epithelium contains the transmembrane mucin (glycosylated glycoproteins present in the glycocalyx), MUC1, MUC4, and MUC16. Mucin's are high molecular weight Glycoproteins that provide a protective layer on corneal epithelial surfaces and are involved in cell-cell interactions, signaling, and metastasis. MUCIN 1, a cell surface associated (MUC 1) or polymorphic epithelial mucin (PEM) is a mucin, the protein part of which is encoded by the MUC1 gene in humans. Mucin proteins penetrate the membranes of epithelial cells, on the inner surface of the intestine and other organs. Mucin protects the body from infection by binding to pathogens. These membrane mucins interact with soluble, secreted, gel-forming mucin produced by the goblet cells (MUC5AC) and also with others like MUC2. The lacrimal gland also secretes MUC7 into the tear film. These soluble mucin in the tear film moves freely, (made possible by blinking and electrostatic repulsion from the negatively charged trans membrane mucin), functioning as clean-up proteins (picking up dirt, debris, desqamated cells and pathogens), holding fluids because of their hydrophilic nature, and harboring defense molecules produced by the lacrimal gland. Transmembrane mucin provide a smooth lubricating surface, allowing lid epithelia to glide over corneal epithelia with minimal friction during blinking and other eye movements. It also prevents pathogen adherence and their entrance on eye bulb. Mucin deficiency is caused by damage to the goblet cells (chemical burns) or the epithelial glycocalyx such as in Stevens-Johnson syndrome. SJS is the result of a severe allergic reaction to a medication, particularly anti-inflammatory medications (NSAIDs), anti consultants such as Dilantin®, Lamictal®, and antibiotic drugs like Allopurinol. It leads to poor wetting of the corneal surface with subsequent desiccation and epithelial damage, even in the presence of adequate aqueous tear production. Our invention of Insulin to be used in dry eye syndrome is effective in inducing goblet cells of the eye lids for producing mucin thus eliminating the symptoms of dry eye syndrome.

Androgen and estrogen receptors are located in the lacrimal and meibomian glands. It has been shown that in meibomian gland dysfunction, a deficiency in androgens results in loss of the lipid layer, specifically triglycerides, cholesterol, monounsaturated essential fatty acids (e.g., oleic acid), and polar lipids (e. g, phosphatidylethanolamine, sphingomyelin). The loss of polar lipids (present at the aqueous-tear interface) exacerbates the evaporative tear loss. Therapeutic agents decrease in unsaturated fatty acids, leads to thicker, more viscous secretions that obstruct ductules and cause stagnation of secretions. Patients on anti androgenic therapy for prostate cancer contain increased viscosity of meibum, decreased tear break-up time, and increased tear film debris, all indicative of a deficient or abnormal tear film leading to DES. Proinflammatory neurotransmitters also play a role in KCS in production of tears form the glandular system. Normal production of tear proteins, such as lysozyme, lactoferrin, lipocalin, and phospholipase A2, is decreased in KCS. A review on dry eye syndrome and its pathophysiology was published by eMdicine by C Stephen Foster, Erdem Yuksel, Fand Anzaar, and Anthony S Ekong, updated on May 13, 2009, which is incorporated herein.

Dry eye syndrome (DES) usually affects both the eyes. Tears, a watery fluid are mostly produced by lacrimal glands. Without tears, good vision is not possible. DES is a common ophthalmological disorder affecting about one in five Americans (approximately 10-30%), in particular, older than 40 years. An estimated 3.23 million women and 1.68 million men, a total of 4.91 million people, aged 50 years and older are affected in US. Dry eye are greater in the Hispanic and Asian populations than in the Caucasian population. An estimated 30 million people suffer from this condition worldwide. It is very prevalent in the Middle East population due to dry desert extreme weather conditions.

Examples of dry eyes are: patients having immune mediated keratoconjunctivitis sicca (KCS) or dry eye disease or other autoimmune dysfunction of the lacrimal gland, dry eye symptoms of contact lens wearers, autoimmune diseases of other systems of the body, infection, and endocrine disease especially in menopausal women, diabetics, aging individuals and individuals with thyroid disorders. Dry eye syndrome is frequently included as part of Sjogren's syndrome (which is really distinct) and the term denotes inflammation of the cornea and conjunctiva secondary to drying. It is typified by symptoms including dry, irritated eyes, excessively watery eyes, burning and stinging, a foreign body sensation, and blurred vision. In all dry eye conditions, the ocular surface epithelium undergoes squamous metaplasia, evidently by loss of goblet cells, mucin deficiency, may be reduced lacrimal and oily gland (meibomian glands) secretion, and keratinization. These changes result instability of the tear film covering the cornea, sclera and the conjunctival sac leads to the clinical symptoms of dry eye syndrome. Human tears are produced by the lacrimal glands which are distributed by blinking which extends aqueous tear film over the ocular surface. They undergo evaporation from the ocular surface, and drain through the nasolacrimal duct (FIG. 1).

The patients with SS associated Dry eye syndrome may be complicated by sterile or infectious corneal ulceration. Ulcers are usually less than 3 mm in diameter, located in the central or paracentral cornea and infrequently may result in corneal perforation and can cause blindness. Other complications include punctate corneal epithelial defects (PEDs), corneal neovascularization, and corneal scarring resulting in visual defects.

In spite of the diverse causes of dry eye syndrome, it is known generically as keratoconjunctivitis sicca (KCS). Dry eyes syndrome can also be seen in Sjogren's syndrome—an autoimmune disease which causes damage to the lacrimal gland. It results in disabling of the reflex aqueous tear production process. Meibomian gland dysfunction (MGD) alters the oily layer in tears, causing increased evaporation resulting in dry eye syndrome. A growing body of research suggests that the dry eye is the result of an underlying cytokine and receptor-mediated inflammatory process. The symptoms associated with dry eye are often exacerbated with subjects using contact lenses. In some cases, contact lens intolerance is caused by the condition of dry eye. The rate of evaporation from the eye is accelerated by the contact lens surface. An aqueous tear film extends over the ocular surface and maintains the ocular surface moist and lubricated which is deficient in dry eye syndrome.

Eye Lid Glands, Their Role in Dry Eye Syndrome and How the Therapeutic Agents of the Present Invention Cure or Curtail the Disease Afflicted Eye Lid Glands

Eye lids contain multiple complex glands whose function is to keep the cornea and conjunctiva healthy with constant supply of secretions, nutrition's, and bacteriostatic agents at the same time to dislodge the foreign bodies from the eye with outpouring of secretions and blinking lids. The glands embedded in the eye lids and fornixes involved in health of the exposed eye ball are: 1. lacrimal glands (57 total), 2. Glands of Krause, 3. Goblet cells, 4. Glands of wolfring, 5. Mucus producing crypts of Henle, 6. Meibomian glands, 7. Glands of Zeis, 8. Glands of Moll, 9. Sweat and 10. sebacious glands on the exposed skin of the eye lid (play no role in tear production). It is important to note that the eye lids, for its size, have more different kinds of secretory glandular system than any other tissue in the body. Any therapeutic agents used to treat dry eye syndrome should have ability to improve and restore the function of these glands, and our invention provides such therapeutic agents.

There are 3 Types of Tears Produced by the Glands of the Eye, Mainly by Lacrimal Glands. They Are:

1. Basal tears: In healthy eyes, the cornea and conjunctival lining is constantly kept wet and nourished by basal tears. They lubricate the eye, and keep it clear of any particulate foreign material such as dust. Tear fluid: The aqueous component is produced by the lacrimal glands. This component includes about 60 different proteins, electrolytes, and water. Lysozyme is the most abundant (20-40% of total protein in tears) and alkaline protein present in tears. It is a glycolytic enzyme that is capable of breaking down bacterial cell walls. Lactoferrin has antibacterial and antioxidant functions. The epidermal growth factor (EGF) plays a role in maintaining the normal ocular surface and in promoting corneal wound healing. Albumin, transferrin, immunoglobulin A (IgA), immunoglobulin M (IgM), and immunoglobulin G (IgG) are also present. It also contains water, mucin, lipids, lysozyme, lactoferrin, lipocalin, lacritin, immunoglobulin's, glucose, urea, sodium, and potassium. Some of the substances in lacrimal fluid (such as lysozyme) fight against bacterial infection as a part of the immune system. Lysozyme does this by dissolving the outer coating of certain bacteria (bacteriolytic). It is a typical body fluid with a salt content similar to blood plasma. Usually, in a 24-hour period, 0.75 to 1.1 grams (0.03-0.04 ounce avoirdupois) of tears are secreted. With advancing age, its production slows; more so in menopausal woman.

2. Reflex tears: The second type of tears results from irritation of the eye by foreign particles, presence of irritant substances such as onion vapors, tear gas or pepper spray, bright light; hot peppery stimuli to the tongue and mouth and during vomiting.

3. Crying or weeping or emotional psychic tears: The third category is increased lacrimation due to emotional stress, suffering, mourning, or physical pain. Emotional tears contain more of the protein-based hormones prolactin, adrenocorticotropic hormone, and leucine enkephalin (a natural painkiller) than basal or reflex tears. The limbic system is involved in production of basic emotional drives, such as anger, fear, sad events, tragic episodes etc. The parasympathetic system controls the lacrimal glands via the neurotransmitter acetylcholine through both the nicotinic and muscarinic receptors. When these receptors are activated, the lacrimal gland is stimulated to produce tears under psychic stimuli.

Medications that causes dry eye syndrome are many and they can be prescription or over-the-counter (OTC), medications. They are: Diuretics, drugs commonly used to treat high blood pressure; Angiotensin-converting enzyme (ACE) inhibitors used to treat high blood pressure; Antihistamines, and decongestants; Sleeping pills; Birth control pills; Certain antidepressants; Isotretinoin-type drugs (a form of vitamin A) for treatment of acne; beta-blockers, phenothiazines, anticholinergics, (atropine, belladona compounds), oral contraceptives, anxiolytics, antiparkinsonian agents, diuretics, anti arrhythmias agents, topical preservatives in eye drops, topical anesthetics, opiate-based pain relievers such as morphine, codeine and many more.

Dry eyes can be caused and worsened by exposure to many environmental conditions that have a drying effect, such as sun, wind, high altitude, dry climate, desert conditions, hot blowing air, dusty working conditions and the cabins of commercial airplanes. When the blinking is reduced such as in driving, neuromuscular diseases affecting the eye lids, watching TV or working on the computer increasing tear evaporation eye dryness. Transient symptoms of dry eye associated with refractive surgery have been reported in some cases from six weeks to six months or more following surgery.

Dry eye syndrome is classified (Schimer score) based on severity, signs and symptoms as 1. Mild and/or episodic; occurs under environmental stress, 2. Moderate episodic or chronic, stress or no stress, 3. Severe frequent or constant without stress and 4. Very Severe and/or disabling and constant.

Signs and symptoms of dry eyes, may include: A stinging, burning or scratchy sensation in the eyes, stringy mucus in or around your eyes, Increased eye irritation from smoke or wind, eye fatigue after short periods of reading, Sensitivity to light, Difficulty wearing contact lenses, tearing, blurred vision, often worsening at the end of the day or after visually focusing for a prolonged period on a nearby task such as focusing on computer screens.

The Eyelids: Anatomy, Histology; Their Roll in Protecting the Eyes with their Glandular Secretion and Blinking in Spreading the Secretions to Prevent Dry Eye Syndrome

The anatomy of the eyelid is described in detail in many texts, including a description in The Anatomy of the Eye and Orbit, Eugene Wolff, The Blakiston Company, Philadelphia, 1948: 140-94 and Grays Anatomy. Understanding of the eye lid structure is important to understand the dry eyes syndrome, and how our invention works in the treatment of dry eye syndrome. Simply because, the eye lid plays a major role in production of mucin and oily secretions which in conjunction with tears from lacrimal glands form a layer covering the exposed cornea and sclera of the eye ball. The eyelids protect, nourish and sustain health of the cornea and scleral covering of the exposed eye ball (FIGS. 1-3). From without, inwards, each eyelid consists of: skin, subcutaneous areolar tissue, fibers of the orbicular oculi, levator palpebrae, smooth muscles (See Table 1), tarsus plates and orbital septum, tarsal glands and conjunctiva. The upper eyelid contains, in addition, the aponeurosis of the levator palpebrae superioris, orbicularis oculi and muscles of Muller (Table 1) helps to move the lid glandular secretion all over the exposed eye ball evenly (see FIG. 1). The skin is exceptionally thin (akin to labia minora and prepuce of the penis) and continuous at the margins of the eyelids with the conjunctiva (see FIGS. 1-3). The subcutaneous areolar tissue is lax, delicate, and seldom contains any adipose tissue. That is why in obese people, the fat in the eye lids is still sparse compared to the rest of the body.

TABLE I
Muscles in the eye lids, their function (action) in
blinking and spreading lid and lacrimal gland secretions
prevent the DES and removing the foreign body.

| | | | Function (action) of the |
|---|---|---|---|
| | | Innervations of these | muscles as related to |
| | Muscle of the | muscles of the | eye lid movement, |
| # | eye lid | eye lid | and blinking |
| 1 | Orbicularis | Somatic fibers from | Blinking/closing the eyelid |
| | oculi | facial (CN VII) | Protects the eye from |
| | (skeletal) | nerve | foreign material from |
| | | | the eye. Helps to |
| | | | spreads the |
| | | | secretions evenly on |
| | | | the exposed eye |
| 2 | Levator | Somatic fibers from | Elevating the eyelid (as |
| | palpebrae | occulomotor | when looking up), |
| | superioris | (CN III) nerve | also contributes to |
| | (skeletal) | | holding the eyelid |
| | | | open and participates |
| | | | in blinking |
| 3 | Superior tarsal | Sympathetic fibers | Helps in holding the eyelid |
| | muscle of | from superior | open (Ptosis if |
| | Muller | cervical | paralyzed) |
| | (smooth | ganglion form | automatically and |
| | muscles) | the neck | provides tone to |
| | | through the | muscles |
| | | carotid blood | |
| | | vessels | |

Forceful or voluntary contraction or squeezing of the Orbicularis oculi of the yes lids can express the glandular secretions of all glands which act as wetting agents.

The upper eyelid is the larger and more movable, due to the levator palpebrae superioris, Orbicularis oculi and smooth muscle of Muller (FIG. 1, TABLE 1). The two eyelids are united to each other at their medial and lateral ends, and when the eye is open an elliptical space, termed the palpebral fissure, their margins join at the angles (canthus) of the eyes are noted. The eyelids or palpebrae are thin, movable folds, tailored to fit the front of the eye ball; protecting it by their closure, from injury and insult by foreign bodies. The lateral angle of the eye (lateral canthus) is more acute than the medial, and lies in close contact with the eyeball. The medial angle (medial canthus) is prolonged for a short distance towards the nose, and is about 0.6 mm away from the eyeball; the two eyelids are here separated by a triangular space, named the lacus lacrimalis, in which a small reddish body, termed the caruncula lacrimalis is situated. On the margin of each eyelid, at the basal angles of the lacus lacrimalis, there is a small conical elevation, termed the lacrimal papilla, the apex of which is pierced by the beginning of the lacrimal canaliculi known as the punctum lacrimale which drains the excess of lacrimal secretions conducting to the nasal cavity.

Each eye lid is covered on its anterior surface with delicate skin; this contains the follicles of very fine hairs (vellus), sebaceous and sweat glands. The edges are lined 3-4 rows of with modified short thick dark terminal hairs called eye lashes. The dermis is a loose texture, and the subcutaneous tissue, deep to it contains almost no fat. The keratin of the epidermis gradually thins out as the skin approaches the free margin of the eyelids, and here the epidermis becomes continuous with the epithelium of the palpebral conjunctiva lining the inner (posterior) side of the lid and the eye ball (FIG. 1).

Each eye lid is reinforced with thin elongated, dense connective tissue tarsal plates (FIG. 1) which facilitates the movement of the eye lid in sweeping motions like a window wipers of vehicles. They are about 2.5 cm long; one is placed in each eyelid and contributes to its form and support. The tarsal plates are placed in the posterior part of the lid so that the palpebral conjunctiva is opposed to its posterior surface. The secretory portions of long, vertically disposed, complex sebaceous glands, called meibomian glands (FIGS. 1,2,3), are embedded in the tarsal plate. They open onto the posterior part of the free margin of the lid behind the eye lashes. They are modified sudoriferous glands also termed ciliary glands, are arranged in several rows close to the free margin of each lid and open behind the attachments of the eyelashes. They produce oily secretions, which mixes with the tears and coats the cornea and bulbar conjunctiva (FIGS. 2, 3).

The upper lid has more Meibomian glands compared to the lower lid, which may account for the greater incidence of sebaceous carcinoma in the upper eyelids. If one of these glands becomes infected, a painful small pea-like swelling develops in the lid called chalazion. The inner lining of the eyelids is conjunctival epithelium, also contains a many goblet cells, located more towards the fornix and less towards at the margin of the lid.

The tarsus of the upper eyelid is a semi-oval form, about 10 mm in height at the center, and gradually narrowing towards its extremities. The lowest fibers of the superficial lamella of the aponeurosis of the levator palpebrae superioris are attached to its anterior surface, and the deep lamella of the same aponeurosis is inserted into its upper margin. The tarsus of the lower eyelid is a narrower and the vertical diameter is about 5 mm. The free or ciliary margins of the tarsi are thick and straight. The attached or orbital margins are connected to the circumference of the orbit by the orbital septum. The lateral ends of the tarsi are attached by a band, named the lateral palpebral ligament, to a tubercle on the zygomatic bone, just within the orbital margin. This ligament is separated from the more superficially placed lateral palpebral raphe by a few lobules of the lacrimal gland. The medial ends of the tarsi are attached by a strong tendinous medial palpebral ligament to the upper part of the lacrimal crest, and to the adjoining part of the frontal process of the maxilla.

The orbital septum is a weak membranous sheet, attached to the edge of the orbit, where it is continuous with the periosteum. In the upper eyelid, it blends with the superficial lamella of the aponeurosis of the levator palpebrae superioris. This muscle along with orbicularis oculi plays a major role in blinking and facilitating the even distribution of the secretion over the exposed eyes. The eye lids are perforated by the blood vessels and nerves which pass from the orbital cavity to the face and scalp.

Deep within the skin covering the anterior surface of the lid are bundles of striated muscle fibers of the Orbicularis oculi muscle (see FIG. 1). Some of the collagen fibers from the aponeurosis of the levator palpebrae muscle pass between these bundles to be inserted into the skin that covers the eyelid. Others connect with the tarsal plate, and still others continue toward the margin of the lid in front of the plate. This latter sheet of connective tissue becomes more areolar as it approaches the margin of the lid, which it reaches to form the gray line, a surgical landmark of some importance. Along this gray line, the lid may be split surgically, opening up the sub muscular space known to the ophthalmologist as the inter marginal space.

The hairs line: the free margins of the eye lids are lined with the eye lashes attached in the free edges of the eyelids from the lateral angle of the eye to the lacrimal papillae. They are short, thick, curved hairs, arranged in 2, 3 or 4 rows in front of the gray line. Each upper eye lid contains 100-125 eyelashes though much fewer on the lower eyelid. The eye lashes are long and thick on the upper lid compared to lower lid lashes. The upper eye lashes curve upwards; those of the lower eyelid curve downwards and outwards so that the upper and lower eyelashes do not interlace when the lids are closed. The hair follicles of the eyelashes slant anteriorly as they pass to the surface. They are provided with sebaceous glands, named the glands of Zeis. Between the follicles, the sweat glands of Moll are disposed (FIGS. 1-3). A sty is the result of the infection of either type of gland.

The palpebral fibers of the Orbicularis oculi muscle are thin (FIG. 1, Table I), pale in color and parallel with the palpebral fissure. Deep to the muscle there is a layer of loose areolar tissue (FIG. 1), which, in the case of the upper eyelid, is continuous with the sub aponeurotic layer of the scalp, so that effusions of fluid (blood or pus) in this layer of the scalp can pass down into the upper eyelid. It is in this layer of the eyelids that the main nerves lie, so that local anesthetics have to be injected deep to the orbicularis oculi.

The tarsal glands are embedded in the thickness of the tarsi, and may be visible through the conjunctiva on everting the eyelids; they present an appearance like parallel strings of pearls (FIG. 1, 2, 3). They are yellow in color, arranged in a single row, and number about thirty in the upper eyelid, and rather fewer in the lower lid. They are embedded in grooves on the deep surfaces of the tarsi and correspond in length with the breadth of these plates; they are, consequently, longer in the upper than in the lower eyelid. Their ducts open on the free margins of the lids by minute foramina. The tarsal glands are modified sebaceous glands, each consisting of a straight tube with numerous small lateral diverticla (FIGS. 1-3). The tubes are supported by a basement membrane and are lined at their mouths by stratified epithelium; the deeper parts of the tubes and the lateral offshoots are lined by a layer of polyhedral cells. The oily secretion of the glands spreads over the margin of the eyelid and tends to prevent the tears from overflowing on to the cheek. It also spreads over the external surface of the tear film and reduces evaporation of the tears form the eye ball coating.

Tears are produced by the lacrimal gland and several accessory tear glands (TABLE II and III) at the upper part of the upper eye lids called glands of Krause. There are a total of 57 glands in each orbit: 1 lacrimal gland+50 glands of Krause (42 in the upper fornix and 6-8 in the lower fornix)+5 glands of Wolfring+1 Caruncle=57 (FIG. 2, TABLE II). The main and accessory lacrimal gland lies in the superolateral corner of the bony orbit. Less than a dozen ducts run from the gland to empty along the superior fornix. Small accessory tear glands, the glands of Krause are scattered along both fornices. Still smaller glands are present in the caruncle. It is of interest that the eye may remain healthy in the absence of the lacrimal gland. This suggests that the function of the gland is to some extent that of providing floods of tears on special occasions and other accessory glands can provide the same lubrication and wetting as the main lacrimal glands. The secretion of the tear glands is slightly alkaline (>pH7). In addition to various salts, it contains an enzyme, lysozyme, which is bactericidal. Tears spread evenly over the cornea and the conjunctiva by the blinking of the lids, which keep the surface of the cornea and the conjunctiva moist. Floods of tears assist in washing foreign particles from the conjunctival sacs and the cornea.

TABLE II
Glands in the eye lids and their contribution to the tears

| | | Their contribution to tear to |
|---|---|---|
| | | form ocular film covering the |
| # | Glands in the eye lids | exposed eye ball |
| 1 | Main Lacrimal glands at the upper | Secret Serous: watery |
| | lateral end (Parasympathetic | secretions containing |
| | nerve supply from the lacrimal | electrolytes, ions, |
| | nucleus of the facial nerve in the | and some proteins |
| | pons), accessory glands of | such as lysozyme and IgA |
| | Krause at the fornix, and glands | |
| | of wolfring on the lid conjunctiva | |
| 2 | Conjunctival goblet cells at the | Secretes Mucous: viscous |
| | upper part of the eye lid | mucopolysaccharides |
| | conjunctiva, and crypts of Henle | complex |
| | and corneal mucopolysaccharides | |
| 3 | Tarsal (Meiboinian) glands | Secrete Sebaceous material |
| | embedded in the tarsal plates | which contains lipids and |
| | and glands of Zeis of the eye | keeps the eyes wet and |
| | lids | facilitate gliding movement |
| | | of the lids |
| 4 | Glands of Moll at the root of the | Their secretions do not |
| | eye lashes and sweat and sebaceous | contribute to the tear to |
| | glands on the skin surface of the | coat the conjunctival sac, |
| | lid | and cornea of the eye ball |

The tears covering the cornea and conjunctiva encompass three layers (Table III): 1. A layer of mucin, a slimy substance produced by the goblet cell and it coats the corneal epithelium, 2 The aqueous tear layer, produced by the lacrimal glands and just about 0.9% saline, floats on the mucin layer, which is in turn covered by an extremely thin (0.01-0.22 μm) layer of lipid molecules. 3. Outside the aqueous tear layer is an oil layer produced by tarsal glands and glands of Zeis located in the eyelid. This layer of an aqueous-lipid—mucin mixture, forms a thin, fine film which floats on top of the tears and limits its evaporation.

TABLE III
Summary of 3 pre corneal and global scleral tear films,
the glands and cells of the eye lids secretion contributes
to them, their content and function.

| Name of | | | |
|---|---|---|---|
| the tear | | Secretor glands | Function of these |
| layers on | Constituents' | which con- | secretion from the |
| the eye | of the ocular | tribute to | eye lid and |
| ball. | tear layers | the tear layers | lacrimal glands. |
| 1. | Oils | Meibomian | It coats the aqueous |
| Superficial | | glands (or | layer; provides a |
| Lipid layer. | | tarsal glands) | hydrophobic barrier |
| 0.11 μm | | and glands of | that impede |
| thick | | Zeis behind | evaporation |
| | | the eye lashes | and prevents tears |
| | | | spilling onto the |
| | | | cheek. These |
| | | | glands are found |
| | | | among the tarsal |
| | | | plates. Thus, |
| | | | the tear fluid |
| | | | deposits between |
| | | | the eye proper |
| | | | and oil acts as |
| | | | barriers of the |
| | | | lids spill over. |
| 2. Middle | Water and | Lacrimal gland, | Promotes spreading of |
| Aqueous | other | (reflex tearing) | the tear film; promotes |
| layer, 0.7 | substances | glands of | the control of infectious |
| μm thick | such as | Krause and | agents; uphold osmotic |
| | proteins | Wolfring | regulation, expresses |
| | (e.g. Tear | (basic tearing) | emotional status (crying). |
| | lipocalin, | | |
| | lactoferrin, | | |
| | lysozyme and | | |
| | lacritin) | | |
| 3. Inner | Mucin | Multiple | It is the hydrophilic |
| most | | conjunctival | quality that allows the |
| Mucous | | goblet cells, | aqueous tears to spread |
| hydrophilic | | crypts of | and coats the cornea and |
| layer. 0.02- | | Henle and | conjunctiva. It allows for |
| 0.05 μm | | ocular surface | even distribution of the |
| thick | | epithelium | tear film on the exposed |
| | | via its loose | eye ball surface |
| | | attachments to | |
| | | the glycocalyx | |
| | | of the micro- | |
| | | plicae of | |
| | | the epithelium. | |

In many patients with dry eye syndrome, the function of the lacrimal glands may be normal, with adequate aqueous tear production; it is one of the other tear layers (Table III) described above which is inadequate. If the lipid layer of the tear film is disturbed by, for example due to trauma, disease, irritation of the eye or contact lens wear, excessive evaporation of water from the eye may occur, leaving the surface of the eye dry leading to dry eye syndrome. The presence of a continuous tear film is important for the health of the corneal and conjunctival epithelium. It provides the cornea with an optically high quality surface for entry of visual light to the retina. In addition, the aqueous part of the tear film acts as a lubricant to the eyelids during blinking of the lids. Enhancement of the function of these other glands, or supplying the deficiencies exhibited by the glands, was not satisfactorily addressed in the prior studies and ophthalmic eye drops to treat DES. The majority of dry eye syndromes are treated with the topical application of eye drops frequently. Our invention enhances the functioning of these glands leading to normal tear production restoring the coating of the exposed cornea and conjunctival sac of the sclera.

Blinking of the Eye Lid and Its Role in Health and Dry Eye Syndrome

Blinking plays a major role in maintaining the health of the eye (Table III) and preventing the dry eyes by evenly coating the cornea and eye ball. The upper eyelids protect the eye. The lower lid is considered essentially stationary and it moves slightly upward movement towards the nose. The role of the upper lid includes:
i. Protection of the eye by emergency closure;
ii. Protection of the eye during sleep; and during blinking,
iii. The spreading of tears across the ocular surfaces,
iv. The wetting of the ocular surfaces by its secretions,
v. The applying of oil from the oil glands (meibomian glands and glands of Zeis), and the spreading of this oil over the surface of the cornea and conjunctiva of the exposed eye,
vi. The removal of foreign matter from the exposed eye ball by physical movement,
vii. Polishing and maintaining the optical surface of the cornea for optimal vision.
viii. Blinking is crucial for comfort, vision, proper functioning of the eye, formation and maintenance of the tear film on the cornea and conjunctiva.
   It is recognized that if the cornea is not sufficiently protected by an adequate tear film, its spread by the movement of the eye lids, the epithelial cells and their tight junctions give in, then subject to a host of obstacles including infection. If the upper lid is unable to close specially during sleep, the consequences are that the epithelial cells of the cornea and the other exposed surfaces of the eye desiccate resulting in discomfort, tearing, and pain and, in severe condition damage to the epithelial cells and deeper tissue of the cornea, ulcerations, even the possible loss of the eye sight.

The blinking which is responsible for spreading of the tear on the eye ball evenly is controlled by the muscles listed in the table I and their nerve supply in the eye lid. Any of the muscles listed in the table I in the eye lid can be affected due to their nerve supply afflictions and interruptions, diseases of the motor ends plate of the muscles, infection resulting in the eye lid dysfunction, muscle and glandular pathology, leading to dry eye syndrome due to lack of eye lid movement (muscle spasm, paralysis, Ptosis, entropion or ectropion due to lid diseases etc.).

The average blink rate in human is about 12 blinks per minute and can vary between 3-5 blinks to as many as about 30 blinks per minute. At the rate of 12 blinks per minute, a person blinks 11000 blinks per day and 4 million per year. (Ploman; The physiology of the eye and vision. In: Duke-Elder S., ed. System of Ophthalm % gy, Volume IV, St. Louis, Mo.: Mosby 1968: 419; York M, Ong J, Robbins J C. Variation in blink rate associated with contact lens wears and task difficulty. AM J Optom Arch Am Acad Optom 1971; 48:461-6; Carney L G, Hill R M; The nature of normal blinking patterns. Acta Ophtha/mol (Kbh) 1982; 60:427-33).

The anatomic histology of the eyelid relevant to the subject of invention is that portion of the upper lid in contact with ocular surfaces is explained in detail which U.S. Patent Application Publication Number: 2007/0036726 Al which is incorporated herein. This portion of the lid may be visualized as a wiping surface roughly analogous to the wiping edge of an automobile windshield wiper blade with water spray. This is the portion of the back surface of the upper eyelid edge that makes direct contact with the ocular surfaces-the cornea and the bulbar conjunctiva. It can only be seen when the upper lid is everted.

This area of the lid is covered with multiple layers squamous epithelial cells like creating a thick band (FIGS. 1-3) and is named as “lid wiper” portion of the eyelid. It makes contact with the lower eyelid during blinking or lid closure as the marginal area, starting in the area of the eyelashes and extending backward to the eye where it is noted that a much sharper junction is formed against the surface of the eye. From the edge of the lid, the multilayered squamous epithelium of the lid wiper changes from the squamous type of epithelial cell to transitional and then to columnar.

The area of the upper lid, which has columnar cells, is not in contact with the ocular surfaces; the space between the columnar cells and the ocular surfaces is termed Kessing's space 438 as shown in FIG. 3. The lid wiper portion of the eyelid cannot be readily observed as it is behind the upper lid margins and tarsal glands opening (FIGS. 1,2,3). The original assumption that only the marginal area made contact with the ocular surfaces appears to have originated in the 1904 publication of Parsons J H, The Pathology of the Eye, Vol. I, Hodder and Stoughton, London, 1904, where Parsons assumed that, owing to the thick squamous type of epithelium in the marginal areas.

This part of the eyelid was in particularly close contact with the eye ball surface, similar to squamous cells of anatomical parts of the body that are designed to make contact. It is believed that the only investigation of the nature of the contact of the inner aspects of the upper lid with the ocular surfaces was conducted by Kessing, (Kessing S Vitiligo. A new division of the conjunctiva on the basis of x-ray examination, Acta Ophthalmologica, Copenhagen, 1967; 45:680-83). Kessing recognized that only the marginal area of the upper eyelid was in contact with the eyeball, while for the lower eyelid, the entire inner area was in close contact with the eyeball. The upper lid above the thick margin does not make contact with the eye ball.

It is recognized that the eye is covered with a complex tear film as described above (Table III). The tear film and eye lid blinking protects the cells of the eyeball from drying, damage from the foreign bodies and cellular debris. As discussed above, blinking is required to cause secretion from the oil glands and to spread the complex tear film over the ocular surfaces to prevent drying. If blinking does not renew the tear film, the cells on the ocular surface, the cornea, and the bulbar conjunctiva, will dry and signal actual damage. Damage resulting from dry eye syndrome can be evaluated by fluorescein or rose Bengal staining agents instilled into conjunctival sac. These stains do not adhere to the healthy cells of the conjunctiva and cornea. They stain or color compromised cells.

The tear film, the cells covering the cornea and the ocular surfaces are examined with the magnification of a slit-lamp utilizing filters to intensify the natural fluorescence of these dyes after one minute after application of the stains. The damage to the tissue is revealed as “staining”, which is the infiltration of the dye into the cell or between the tight junctions of the cells.

The position of the lid wiper on the upper eyelid and the location of the squamous cells are Illustrated in publication U.S. Patent Application Publication Number: 2007/0036726 A1 which are incorporated here in. The lid margin which acts as wiper is the small area that would be in e contact with the ocular surfaces as the upper lid moves up and down during blinking. In use, it is separated from the ocular surfaces by a boundary layer of tear fluid. It is thought that this border state line tear fluid could be as thin in the range of 5 to 10μ thick. The marginal conjunctiva, and the lid wiper are covered with multiple layers of squamous epithelium (FIG. 1-3), a type of epithelium designed for contact akin to skin.

As the epithelium continues upward on the inner surface of the lid from the area of the lid wiper, it changes from the squamous type of epithelial cell to transitional and then to columnar. The area of the upper lid, which has columnar cells, is not in contact with the ocular surfaces, the space between the columnar cells and the ocular surfaces is named as Kessing's space.

Test for Dryness of the Eye in the Dry Eye Syndrome

The presence of dry and irritation of the eyes can test by ophthalmologist. They can test both the quantity and the quality of your tear by measuring your tear production using the Schirmer tear test. In this test, blotting strips of paper are placed under your lower eyelids under topical anesthesia. After five minutes your doctor measures the amount of strip soaked by your tears. Other tests use special dyes in eye drops to determine the surface condition of your eyes specially for staining patterns on the cornea and measures how long it takes before your tears evaporate.

Present Treatment Modalities of Dry Eye Syndrome

Tears like any other fluid will evaporate when exposed to air. The following simple steps can reduce the chances of full blown dry eye syndrome and they are easy to follow. They are:
1. Avoid air blowing in your eyes by use of direct hair dryers, car heaters, air conditioners or fans toward your eyes.
2. Wear glasses on windy days and goggles while swimming
3. Add moisture to the air in winter by a humidifier to add moisture to dry indoor air.
4. Use specially designed glasses that form a moisture chamber around the eye, creating additional humidity worn day and night. Wear them if you sleep with their eyes partially open.
5. Avoid rubbing your eyes which can irritate your eyes and avoid smoking.
6. Use eye drops before, rather than after, your eyes become irritated as a result of visually demanding activities.
7. Consciously blinking repeatedly helps to spread tears more evenly.

When performing tasks that require intense visual concentration, take occasional breaks adding up to about 3-5 minutes each hour and rest your eyes by closing your lids for several seconds with light squeeze which will help to expel the secretions from the eye lid glands into the conjunctival sac.

The treatment of dry eye involves supplement and stabilize the ocular tear film by using artificial tears instilled throughout the day. Examples of the tear replacement approach include the use of buffered, isotonic saline solutions, aqueous solutions containing water soluble polymers that render the solutions more viscous and thus less easily shed by the eye. Tear reconstitution is also attempted by providing one or more components of the tear film such as phospholipids oils (McCulley and Shine, Tear film structure and dry eye, Contactgia, volume 20(4), pages 145-49 (1998); Shine and McCulley, Keratoconjunctivitis sicca associated with meibomian secretion polar lipid abnormality, Archives of Ophthalmology, volume 116(7), pages 849-52 (1998)).

Most people have suffered from dry eyes at some point in their lives; however, there are some who suffer from it more often who need intervention. Unfortunately, most people will reach for over the counter treatments to try and relieve dry eyes but the relief is temporary and may make the condition worst. Over the counter medication, Visine only constricts the blood vessels and but does not play any role in relieving dry eye syndrome.

Dry eye syndrome can be relieved by the use of natural eye drops such as Viva drops or Similasan which have no preservatives, which is also temporary. Taking 1500 mg of primrose oil containing fatty acid in the evening can be therapeutic by increasing the tear production. Oral intake of Vitamin C, GLA, omega 3, and vitamin B6 can be of help who suffer from dry eye syndrome and contact lens users. Eating banana a day can be helpful, because it contains potassium which will reduce the dry eyes symptoms.

There are many methods of treatment of dry eye syndrome which include the following: Artificial tear substitutes, Gel/ointment, Moisture chamber spectacles; Anti-inflammatory agents (Topical cyclosporine a, corticosteroids, topical/systemic tetracycline's). Topical/systemic omega-3 fatty acids inhibit the synthesis of the lipid mediators and block the production of IL-1 and TNF-alpha. The ideal artificial lubricant should be preservative-free and non allergic.

The simplest ophthalmic drops are a wetting and lubricant agent containing potassium, bicarbonate, other electrolytes, and have a polymeric system to increase its retention time. Artificial tears contained hydroxypropyl methylcellulose (HPMC), carboxyl methylcellulose (CMC), polyvinyl alcohol (PVA), glycerin are used to increase lubrication of the eye.

Punctal plugs are also used to prevent the drainage of eye secretion. There are many kinds, made up of collagen, polymers, silicone, and a thermo sensitive, hydrophobic acrylic polymer. There are oral medications used to enhance the lacrimal secretions called Secretagogues-Diquafosol (INS365, DE-089)-P2Y2 receptor agonist; Autologous/umbilical cord serum.

Another drug available is an organo sulfur compound, anethole dithiolethione (ADT, 5-[p-methoxyphenyl]-3H-1,2-dithiol-3-thione, trade name Sialor™) with hardly any side effects. It stimulates the secretion of saliva in patients with autoimmune exocrinopathy (e.g. Sjogren's syndrome). Sialor alleviates the symptoms of xerostomia and xeroophthalmia. We have used Sialor, 25 mg orally and/or ophthalmic drops with successes in these conditions especially those on chemotherapy, menopausal women, and chronic smokers with dry mouth and dry eyes conditions. ADT increases the glutathione synthesis by activating gamma-glutamyl-cysteine synthetase. ADT reduced both polymorphonuclear neutrophils adhesion to ROS (reactive oxygen species—can be effective in post perfusion adhesion of white blood cells to ROS with resultant damage) and stimulated tyrosine phosphorylation.

ADT increased redox status by increasing intracellular glutathione (GSH) content in oxidized cells. These results show that GSH can reverse the effect of oxidation on tyrosine kinase activation and phosphorylation, and thus plays an important role in cell signaling, which confirm the antioxidant activity of ADT. This can be one of the important non toxic oral and eye drops for the treatment of dry eye syndrome and Sjogren's syndrome. (Ben-Mandi M H, Gozin A, Driss F, Andrieu V, Christen M O, Pasquier C. Anethole dithiolethione regulates oxidant-induced tyrosine kinase activation in endothelial cells. Antioxide Redox Signal. 2000 Winter; 2(4):789-99, Personal observation).

Studies by Han et al show that ADT is more bioavailable aqueous or lipid-based formulations, sub-micro emulsion (SME) and oil solution prepared using short (SCT), medium (MCT) and long (LCT) chain triglycerides respectively (Han S F, Yao T T, Zhang X X, Gan L, Zhu C, Yu H Z, Gan Y. Int. J Pharm. Lipid-based formulations to enhance oral bioavailability of the poorly water-soluble drug anetholtrithione: effects of lipid composition and formulation. 2009 Sep. 8; 379(1):18-24. Epub 2009 Jun. 7). Experimental studies also show that the ADT treatment rises glutathione levels significantly as compared with untreated Wurzburg cells. H₂O₂ induced lipid peroxidation was remarkably inhibited by ADT pretreatment of these cells. ADT, a pro-glutathione antioxidant, was observed to be capable of modulating NF-kappa B activation.

Redox regulation of NF-kappa B has been implicated in the activation of the human immune-deficiency virus (HIV). Therefore, inhibition of NF-kappa B activation may be an effective strategy for acquired immunodeficiency syndrome therapy. (Sen C K, Traber K E, Packer L. Inhibition of NF-kappa B activation in human T-cell lines by anetholdithiolthione. Biochem Biophys Res Commun. 1996 Jan. 5; 218(1):148-53).

Other methods include Systemic immune suppressants; Contact lenses made of silicone, permeable hard contact lenses, and overnight wear highly oxygen-permeable lenses. Surgical methods such as Amniotic membrane transplantation, Lid surgery, Tarsorrhaphy and finally Mucous membrane/salivary gland transplant are also attempted.

There are many examples of phospholipids compositions for the treatment of dry eye syndrome that are disclosed. U.S. Pat. No. 4,131,651 (Shah et al.), U.S. Pat. No. 4,370,325 (Packman), U.S. Pat. No. 4,409,205 (Shively), U.S. Pat. No. 4,883,658 (Holly), U.S. Pat. No. 5,371,108 (Korb et al.) and U.S. Pat. No. 5,578,586 (Glonek et al.). U.S. Pat. No. 5,174,988 (Mautone et al.) disclose the Phospholipid drug delivery systems involving phospholipids, propellants and an active substance.

U.S. Pat. No. 4,818,537 discloses the use of a lubricating, liposome-based composition. U.S. Pat. No. 5,800,807 discloses compositions containing glycerin and propylene glycol for treating dry eye syndrome. U.S. Pat. No. 5,041,434 discloses the use of sex steroids, such as conjugated estrogens, to treat dry eye conditions in post-menopausal women; U.S. Pat. No. 5,290,572 discloses the use of finely divided calcium ion compositions to stimulate pre ocular tear film production. U.S. Pat. No. 4,966,773 discloses the use of micro fine particles of one or more retinoids for ocular tissue normalization.

Cyclosporins are a group of nonpolar cyclic oligopeptides with recognized immunosuppressant action and are used in post allogenic organ transplant. Cyclosporin is thought to bind to the cytosolic protein of T-lymphocytes which in turn is responsible for activating the transcription of interleukin II. It also inhibits Lymphokine production and interleukin release which leads to a reduced function of effector T-cells. It does not affect cytostatic activity (i.e. Inhibiting or suppressing cellular growth and multiplication). It prevents the mitochondrial permeability transition pore from opening, thus inhibiting cytochrome C release, a powerful apoptotic stimulus factor.

In the dry eye syndrome, cyclosporins probably act by inhibiting the inflammation and inflammation related release of cytokines, Lymphokine and interleukins which play a role in dry eye syndrome; without suppressing the cellular growth of the cornea and conjunctiva.

The use of cyclosporine A and its derivatives to treat ophthalmic conditions has been the subject of various patents, for example Ding et al U.S. Pat. No. 5,474,979; Garst U.S. Pat. No. 6,254,860; and Garst U.S. Pat. No. 6,350,442, disclosure of each of which is incorporated herein by reference.

U.S. Pat. Nos. 5,051,402, 5,474,979, 5,981,607 disclose treating symptoms in dry eye patients and contact lens wearers with an emulsion of a higher fatty acid glyceride, polysorbate 80 and an emulsion stabilizing amount of Pemulen® in water suitable for topical application to ocular tissue.

U.S. Pat. No. 4,839,342 discloses that cyclosporin (referred as “cyclosporine” or “ciclosporin”) to be effective in treating keratoconjunctivitis sicca (dry eye syndrome). There are several metabolites from B through I has been identified. The commercially available cyclosporin contains many form of cyclosporins combinations. Cyclosporins share a cyclic peptide structure made up of eleven amino acids with a sum molecular weight of about 1,200 with other amino acids. The solubility of cyclosporin in water is between 20 mug/ml to 30 mug/ml. Hence, the pharmaceutical oily solutions are used as a vehicle.

Unfortunately, the solubility of cyclosporin in water is extremely low and as elaborated in U.S. Pat. No. 5,051,402, practically impossible to prepare a pharmaceutical composition containing cyclosporin dissolved in an aqueous medium due to the separation of cyclosporin as a solid immediately after it comes into contact with water, such as in the mouth or eye of the patients.

U.S. Pat. No. 5,051,402 discloses a method of solubilizing cyclosporin in aqueous solution with alpha-cyclodextrin. A topical emulsion of cyclosporin for treating KCS has been promoted under the trade name Restasis (Allegan, Inc., Irvine, Calif.) formulated according U.S. Pat. No. 5,474,979. It contains cyclosporin is in an admixture with a higher fatty acid glyceride, such as castor oil, and a surface active agent, such as polysorbate 80, and an emulsion stabilizer, such as a cross-linked polyacrylate. However, treatment with an emulsion containing oily droplets can result in eye irritation, burning sensation or a clouding of visual field. Due to oily preparation, the active ingredient is less bioavailable. It should not be used by patients with active eye infections, herpes viral infections and trachomatis.

U.S. Patent Application Publication Number: 2010/0016219 Al describes an ophthalmic composition containing aqueous solubilized cyclosporin for treatment of ophthalmic disorders including keratoconjectivis Sicca and ocular rosacea. These inventors state that the application of an emulsion containing oily droplets may result in eye irritation or a clouding of visual field. Furthermore, active ingredient is generally more bioavailable in water solubilized form than in insoluble, suspended, or inclusion complex form.

There are many biological agents developed and under development to treat the Sjogren's syndrome (Jiska M. Meijer, Justin Pijpe, Hendrika Bootsma, Arjan Vissink, and Cees G. M. Kallenberg The Future of Biologic Agents in the Treatment of Sjögren's Syndrome Clin Rev Allergy Immunol. 2007 June; 32(3): 292-297). Sjogren's syndrome (SS) is a chronic lymph proliferative autoimmune disease with disturbances of T lymphocytes, B lymphocytes, and exocrine glandular cells such as salivary glands and lacrimal glands of the eye (Hansen A, Lipsky P E, Dorner T (2005) Immunopathogenesis of primary Sjogren's syndrome: implications for disease management and therapy. Curr Opin Rheumatol 17:558-565). SS can be primary (pSS) or secondary SS (sSS), the latter being associated with another autoimmune disease [e.g., rheumatoid arthritis, systemic lupus erythematosus (SLE)].

Lymphocytic infiltrates are a characteristic histopathological finding in SS. These infiltrates consist of T and B cells. The expression of different cytokines, such as tumor necrosis factor-α (TNF-α) and interferon-α (IFN-α), during the formation and proliferation of these infiltrates has been investigated. There is an over expression of TNF-α, which is secreted by CD4+ T lymphocytes, mononuclear cells, and epithelial cells (Mariette X, Ravaud P, Steinfeld S, Baron G, Goetz J, Hachulla E et al (2004) Inefficacy of infliximab in primary Sjogren's syndrome: results of the randomized, controlled Trial of Remicade in Primary Sjogren's Syndrome (TRIPSS). Arthritis Rheum 50:1270-1276).

The intra glandular synthesis of TNF-α causes destruction of acini by up-regulation of Fas at the surface of the glandular epithelial cells, stimulation of secretion of type 2 and 9 matrix metalloproteases by epithelial cells, and over expression of different chemokines. IFN-α is produced by activated plasmacytoid dendritic cells in primary SS (pSS), and numerous IFN-α-producing cells have been detected in labial salivary glands. IFN-α promotes the autoimmune process by increasing autoantibody production and through the formation of endogenous IFN-á inducers. IFNs have potent immunomodulating properties and are thought to trigger a systemic biological response (Cummins M J, Papas A, Kammer G M, Fox P C (2003) Treatment of primary Sjogren's syndrome with low-dose human interferon alfa administered by the oro-mucosal route: combined phase III results. Arthritis Rheum 49:585-593).

Besides the presence of Proinflammatory cytokines, recent studies have shown an important role for B cells in the pathogenesis of SS. Presence of auto antibodies and hyper gammaglobulinemia are both considered to reflect B cell hyperactivity. Recent insights in the cellular mechanisms of T and B lymphocyte activity in the pathogenesis of SS and the current availability of various biological agents have resulted in new strategies for therapeutic intervention in SS. The gain in knowledge regarding the cellular mechanisms of T and B lymphocyte activity in the pathogenesis of Sjögren's syndrome (SS) and the current availability of various biological agents (anti-TNF-α, IFN-α, anti-CD20, and anti-CD22) have resulted in new strategies for therapeutic intervention of Sjogren's syndrome affecting the lacrimal glands resulting in dry eyes. The biological agents used in SS trials are IFN-α and agents targeting TNF-α and B cells (anti-CD20, anti-CD22).

There is neither a known medicine for Sjogren's syndrome nor a specific cure to permanently restore glandular secretion. The treatment is symptomatic and supportive such as: Moisture replacement therapies such as artificial tears, use of goggles to increase local humidity, punctal plugs, cyclosporin (Restasis), lacrimal flow, such as cevimeline (Evoxac) and pilocarpine; Nonsteroidal anti-inflammatory drugs, corticosteroids or immunosuppressive drugs such as methotrexate, Hydroxychloroquine (Plaquenil). Multiple monoclonal antibodies and biological agents are currently under investigation (Meijer J M, Meiners P M, Vissink A, Spijkervet F K, Abdulahad W, Kamminga N, Brouwer E, Kallenberg C G, Bootsma H. Effectiveness of rituximab treatment in primary Sjögren's syndrome: a randomized, double-blind, placebo-controlled trial. Arthritis Rheum. 2010 April; 62(4):960-8).

Most of the approaches of treating dry eye syndrome with or without associated Sjogren's syndrome have met with some success, and the problems in the treatment of dry eye nonetheless remain. The use of tear substitutes, are temporarily effective, need repeated application over the course of a patient's waking hours even up to 10 applications over the course of the day. Such an undertaking is not only cumbersome, time consuming, and can be expensive. None of the dry eye syndrome medications are natural human body products or hormone of the body; have physiological role in restoring the disease afflicted cells of the cornea, conjunctiva, glands in the eye lids and tear producing lacrimal glands. Our invention will remedy such a deficiency which is derived from within.

### SUMMARY OF THE INVENTION

It is an object of the present invention to develop a liquid eye drop of therapeutic, pharmaceutical, biochemical and biological agents or compounds composition using insulin to be used as eye drops for treatment of dry eye syndromes due to diverse etiologies.

It is an object of the present invention to develop a liquid eye drop of therapeutic, pharmaceutical, biochemical and biological agents or compounds composition using IGF-I to be used as eye drops for treatment of dry eye syndromes due to diverse etiologies.

It is a further object of the invention to develop an eye drop composition with insulin that may further be used to relieve irritations from the eye and eliminate dryness of the eye.

It is a further object of the invention to develop an eye drop composition with insulin with insulin activity enhancers that may further be used to relieve irritations from the eye and eliminate dryness of the eye.

Another object of this invention is the use insulin for the the dry eye syndrome to develop a treatment modality for patients suffering from compromised squamous epithelial surface of the lid wiper due to repeated attacks of blepharitis and help the antibiotic and antiviral drops in eliminating the infections.

Another object of this invention is the use of the aforesaid diagnosis of dry eye syndrome to develop a treatment modality using insulin for patients suffering compromised squamous epithelial surface of the cornea and conjunctiva. This means that the cells are exhibiting an abnormality or “Defective” epithelial cells indicating the damage to these cells, but not dead cells.

Another object of this invention is the use of the aforementioned diagnosis to develop insulin treatment modality for patients suffering from dry eye syndrome due to autoimmune disease such as Sjogren's syndrome.

Another object of this invention is the use of the abovementioned diagnosis to develop a treatment modality using insulin for patients suffering from dry eye syndrome related to meibomian (tarsal) gland dystrophy (MGD).

One important aspect of this formulation of the invention, for effective treatment, is the provision of the omega-3 fatty acids from both plant and fish sources, due to the synergistic combination of the two types of fatty acids from these difference sources combined with insulin for effective therapy.

In accordance with the present invention, a non-irritating pharmaceutical composition with high comfort level and low irritation potential suitable for delivery to sensitive areas such as ocular tissues comprises an admixture of an emulsifying amount of a higher fatty acid glycerol and polysorbate 80 with various therapeutic, pharmaceutical, biochemical and biological agents or compounds such as cyclosporins. The higher fatty acid glyceride may comprise, for example, castor oil, corn oil, sunflower oil or light mineral oil combined with insulin.

In accordance with the present invention, a non-irritating pharmaceutical composition with high comfort level and low irritation potential suitable for delivery to sensitive areas such as ocular tissues comprises of mixture of cyclosporine with insulin combined.

The objects are accomplished by treating the eye with an aqueous composition containing an effective amount of a nonionic surfactant and insulin.

It is another object of this invention to use insulin along with sex steroids such as estrogens and testosterones especially women and men undergoing hormone therapy.

It is a further object of the invention to develop an eye drop composition with insulin that may further be used to relieve KCS, Sjogren's syndrome, Meibomian gland dysfunction (MGD), and dry eye syndrome using insulin along with other therapeutic agent's already FDA approved such as cyclosporins or non approved eye preparations available as eye drops medicaments.

### BRIEF DESCRIPTION OF THE DRAWINGS

FIG. 1 is a histological preparation of a low resolution microscope cross-section view of the upper eyelids showing the eyelid and its histological components including various glands and muscles which make eyelid structures.

FIG. 2 is a diagrammatic presentation of the eyelid with various glands, modified terminal hairs on the outer surface and vellus hair at the edge of the upper eyelid located in front of the opening of the Meibomian glands. Note the thick squamous cell layer covering the edge behind the tarsal gland opening.

FIG. 3 is a diagrammatic presentation of the eyelid showing the edge of the eye lid covered with multiple layers squamous epithelium and the tarsal glands with their oily secretion carried to the surface of the eye ball with help of blinking of the eye lids. Modified terminal hairs are located in front of the opening of the Meibomian glands.

FIG. 4 is the diagram showing the route of drainage of the lacrimal fluid and therapeutic agents from the conjunctival sac to the nasal mucosa and method to prevent it drainage to the nose to prevent the systemic effects of eye drops.

### DETAILED DESCRIPTION OF THE INVENTION

The final ophthalmic drops or preparations to be used to treat dry eye syndrome and other eye diseases should be stable, dissolved or solubilized, safe, effective, and opthalmologically acceptable. The term ‘stable’, means physical, rather than chemical stability with no crystallization and/or precipitation in the compositions, when stored at a refrigerated temperature and when it comes in contact with lacrimal secretions. The label ‘dissolved’, ‘dissolving’, ‘solubilized’ or ‘solubilizing’, means that an ingredient is substantially solubilized in the aqueous composition without in the particulate, crystalline or droplet form in the composition.

The phrase ‘ophthalmically acceptable’, refers to those therapeutic, pharmaceutical, biochemical and biological agents or compounds, materials, compositions, and/or dosage forms suitable for use in a mammalian eye without excessive toxicity, irritation, allergic response, or other problem or complication, commensurate with a reasonable benefit/risk ratio. The expression ‘safe and effective’, as used herein, means a concentration and composition, that is sufficient to treat without serious local or systemic side effects. The term “ocupopathies” means any and all diseases affecting the eye lids, eye ball and its function.

Any treatment designed to treat dry eye syndrome and other eye diseases including Sjogren's syndrome should encompass the following principles: 1. Eye drops, gels or ointments should act as artificial tear film covering like natural tears over the ocular surface of the eye including cornea with least stinging or burning sensation, 2. Capable of providing mechanical lubrication for the ocular surface so that the eye lid glides easily during the blinking movement, 3. Reduce the evaporation of natural lacrimal fluid, 4. The emulsion or the watery ophthalmic drops should not react with eye cellular structures, its lacrimal coating, and eye lid lacrimal glandular system, 5. Eye drops Should be stable for reasonable period of time at room temperature, 6. Besides being acting as lubricant, it should contain therapeutic, pharmaceutical, biochemical and biological agents or compounds capable of alleviating the underlying cause in the glandular system responsible for the dry eye syndrome; at the same time augment-amplify the effects of this invention.

Our invention insulin is based on meeting the above recited pharmacological and therapeutic parameters.

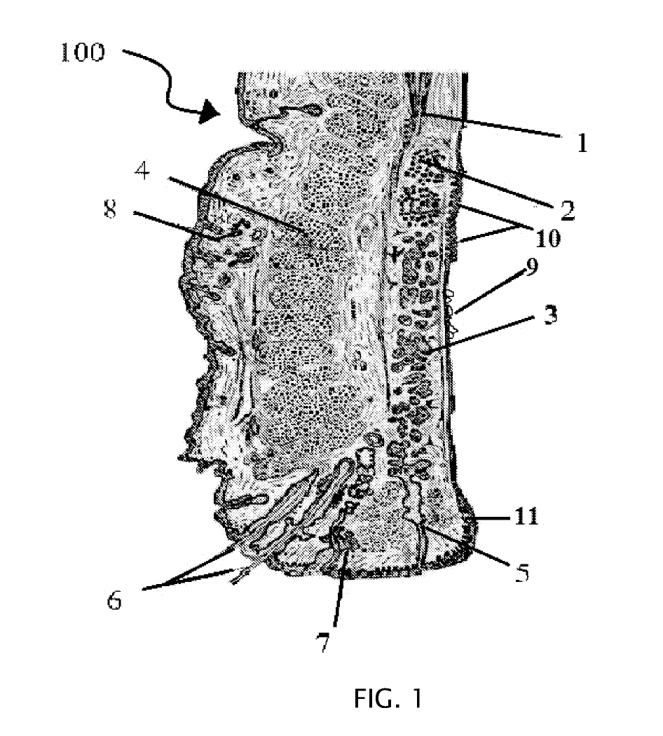

Referring to FIG. 1, a drawing of a low resolution microscope cross-section view of the upper eyelid 100 shows levator aponeurosis (1) enters between the Orbicularis oculi muscle and the conjunctival surface. Accessory lachrymal glands of Wolfring (or Ciacco) are shown (2). The meibomian glands (3) of the tarsal plate produce the lipid that will line the layer of the tear film. The Meibomian lids empty into ducts that dot the marginal surface of the eyelid (5) and can be seen emanating droplets of oil for the tears. The Orbicularis oculi muscle (4) is striated muscle that is responsible for blinking and squeezing eyelids shut and helps to express the glandular secretions when squeezed. The cilia or eyelashes (6) emanate from the lid immediately adjacent to apocrine glands of Moll (7). The skin surface (8) of the eyelid is the thinned epidermis in the body and contains vellus hair and adnexa.

The eye lid inner surface contains multiple goblet cells located at the upper part of eye lid (10). The conjunctiva also contains crypts of Henle (9) on its lining which produce mucin to coat the eye ball. Note the thickening of the edge of the eye lid (11) behind the meibomian glands due to multiple layers of squamous epithelium stacked on each other which comes in contact with the cornea and bulbar conjunctiva, as the upper eye lid moves up and down (blinking action) like car window wiper on the eye ball, spreading the secretions of the eye lid and lacrimal glands over cornea and bulbar conjunctiva evenly (from Grey's anatomy, see tables I, II, & Ill).

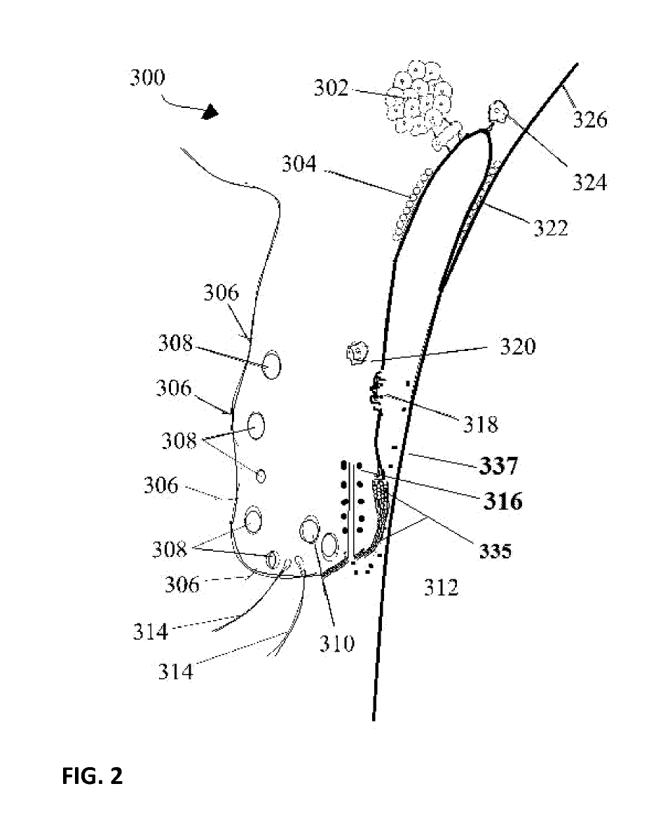

Now referring to FIG. 2, a diagrammatic presentation of the eye lid with various glands 300 in the upper eyelids, modified terminal hairs on the outer surface and vellus hair 306 at the edge of the eye located in front of the opening of the Meibomian glands 316. Also shown are the lacrimal glands 302, goblet cells 304, sweat gland 308, glands of Moll 310, glands of Zeis 312, eyelash 314, crypts of Henle 318, lacrimal glands of Wolfring 320, bulbar conjunctiva 322, lacrimal glands of Krause 324, bulbar conjunctiva 31 and cornea 327. The conjunctiva also contains crypts of Henle 318 on its lining which produce mucin to coat the eye ball. Note the thickening of the edge of the eye lid 335 behind the meibomian glands due to multiple layers of squamous epithelium stacked on each other 435 which comes in contact with the cornea and bulbar conjunctiva, as the upper eye lid moves up and down (blinking action) like car window wiper on the eye ball, spreading the secretions of the eye lid and lacrimal glands over cornea and bulbar conjunctiva evenly. Above these squamous cell bundles, the lid and eye bulb do not come in contact during blinking and this space is called the Kessing's space.

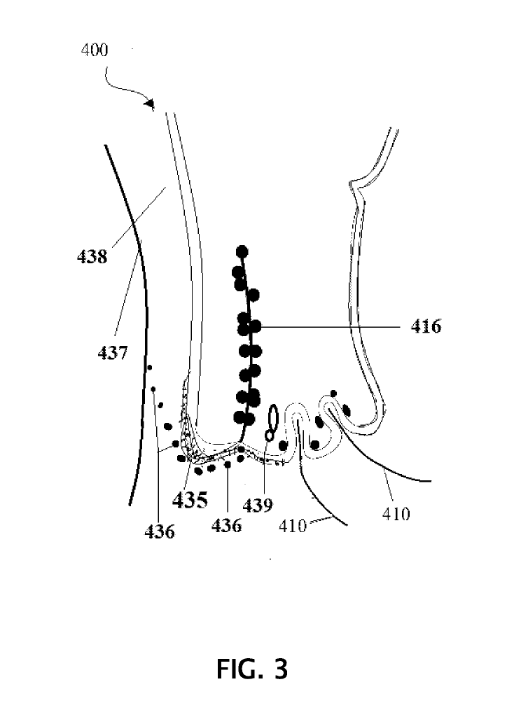

Referring now to FIG. 3, is a diagrammatic 400 presentation of the eyelid margins with rows of eye lashes are presented. It shows the eye lashes 410, meibomian glands 416 and glands of Zeis 439 which secretes oily secretions 436 which coats the cornea and bulbar conjunctiva 437 and prevents the evaporation of the lacrimal coating of the exposed eye ball. Note the thickening of the edge of the eye lid 435 behind the meibomian glands openings due to multiple layers of squamous epithelium stacked on each other which moves the oily secretions of the tarsal glands on to the surface of the cornea and bulbar conjunctiva, as the upper eye lid moves up and down (blinking action) like car window wiper on the eye ball, spreading the secretions of the eye lid glands and lacrimal glands over cornea and bulbar conjunctiva evenly. Above these squamous cell bund, the lid and eye bulb 437 does not come in contact during blinking and this space is called the Kessing's space 438.

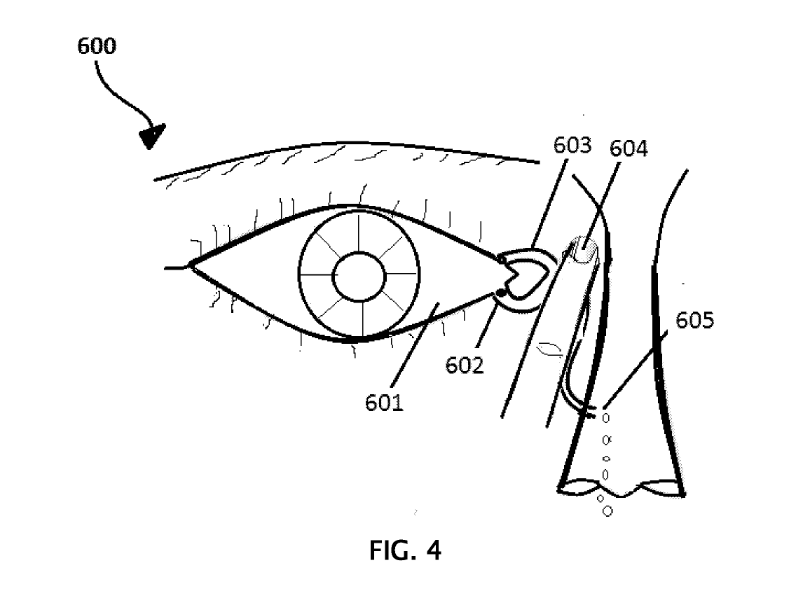

Referring to FIG. 4 is the diagrammatic presentation 600 showing the route of drainage of the lacrimal fluid and therapeutic agents from the conjunctival fornix 601 to the nasal mucosa 605 and method to prevent it. A simple method of applying the finger pressure at the medial eye angle and nasal junction, the location of the lacrimal punctum, canaliculi 602, 603 and lacrimal sac with a finger 604 will prevent the therapeutic agents flow to the nose, and its contact with the nasal mucosa 605, and their associated systemic adverse effects.

Before explanation and description of the disclosed embodiments of the present invention in detail, it is to be understood that the invention is not limited in its application to the details of the particular examples and arrangement shown since the invention is capable of other examples and embodiments in treating other oculopathies. Also, the terminology used herein is for the purpose of description and not of limitation. As earlier enumerated above and recited below; this application has been filed in order to disclose: Insulin and Insulin-like Growth factor (IGF-1) have been found to have high therapeutic activity against dry eye syndrome; also known generically as keratoconjunctivitis sicca (KCS) including Sjogren's syndrome and Meibomian gland dysfunction (MGD).

Dry eye syndrome can also be seen in Sjogren's syndrome caused by damage to the lacrimal gland, which disables the reflex aqueous tear production process due to autoimmune disease. Meibomian gland dysfunction (MGD), alters the oily layer in tears, causing increased evaporation, an aqueous tear film extends over the ocular surface and maintains the ocular surface moist and lubricated. Insulin and/or IGF-I not only restores the proper physiological functioning of the secretory glands of the eye lids, it also enhance the effectiveness (augmentation-amplification effects) of other therapeutic, pharmaceutical, biochemical and biological agents or compounds used in the treatment of dry eye syndrome and other oculopathies in smaller doses than indicated; which in turn reduces or eliminates their systemic adverse effects.

At present, the insulin is exclusively used to treat type I and some cases of type II diabetes. Our discoveries and inventions describes its use topically (locally) in other disease conditions other than diabetes including dry eye syndrome, prostate diseases, middle and inner ear afflictions, CNS diseases, to treat hair loss, enhancing eye lashes, activating vaccines, cytokines, Lymphokine, monoclonal antibodies; activating local immune system at lymph nodes, enhancing the local effects of chemotherapeutic agents, in treatment of autoimmune diseases, age related changes of the facial skin, healing of wounds, gum diseases, local infections and multiple local and systemic therapeutic applications.

Insulin and Its Biological Effects on Healthy and Disease Afflicted Cells (Example: Dry Eyes Syndrome); Its Role in Uptake, Augmentation-Amplification Effects of Therapeutic, Pharmaceutical, Biochemical and Biological Agents or Compounds on these Cells are Described Herein.

A variety of carriers, adjuvant agents, absorption enhancers and facilitators, assist to get entry into the cell, potentiators of therapeutic action (augmentation-amplification effects), cell metabolic activity enhancers, cell multiplication enhancers and other methods have been used to enhance the absorption and/or to potentiate the effect of therapeutic, pharmaceutical, biochemical and biological agents or compounds administered to the patients for improving the physiological function and the treatment of diseases. Discovery of insulin described in this invention is such an agent which we give details and elaborate below.

In 1921, the medical researchers Drs. Frederick Banting and Charles Best at University of Toronto physiology department; isolated insulin from dog pancreas and tested on diabetic dogs, successfully lowering the dogs' blood sugar level. On Jan. 11, 1922, Leonard Thompson, a 14-year-old boy who was dying of diabetes, was given the first human experimental dose of insulin. He lived 13 more years and died at the age of 27 from pneumonia (there was no penicillin at that time yet), not from diabetes.

So far there are no reports of using the insulin as therapeutic agent locally to treat localized diseases or parentarily to treat systemic diseases other than diabetes. The present inventor is the first person to experiment and use insulin locally for almost a decade to treat many kinds of diseases of various tissues and organs in the body including cancers, and diseases of the ear, prostate, teeth, gums, CNS, eyes, hair growth, and other such conditions with many known therapeutic, pharmaceutical, biochemical and biological agents or compounds.

In 1965 Sodi-Pollares et. al. for the first time used glucose-insulin-potassium (GIK) solutions to treat patients with acute myocardial infarction and found that GIK limited infarct size, reduced ventricular ectopy, and improved survival (Sodi-Pollares D, Testelli M D, Fisleder B L. Effects of an intravenous infusion of a potassium-glucose-insulin solution on the electrocardiographic signs of myocardial infarction. Am J Cardiol. 1965; 5:166-81). Insulin benefits the post ischemic myocardium by stimulating pyruvate dehydrogenase activity, which in turn stimulates aerobic metabolism on cardiac and other tissue reperfusion.

Exogenous insulin also helps to reverse insulin resistance during cardiopulmonary bypass, which contributes to increased serum concentrations of free fatty acids and decreased myocardial uptake of glucose and increased myocardial function. Intravenous infusions of insulin after coronary artery bypass graft surgery (CABG) have been shown to decrease the levels of free fatty acids and increase myocardial uptake of glucose. Insulin added to antegrade and retrograde tepid (29° C.) blood cardioplegia during coronary artery surgery has been shown to stimulate aerobic metabolism during reperfusion, preventing lactate release and improving left ventricular stroke work index.

Insulin is especially beneficial for patients with diabetes and acute coronary ischemia (Svensson S, Svedjeholm R, Ekroth R. Trauma metabolism of the heart: uptake of substrates and effects of insulin early after cardiac operations. J Thorac Cardiovasc Surg. 1990; 99:1063-73. Rao V, Mississauga C N, Merrante F. Insulin cardioplegia for coronary bypass surgery [abstract]. Circulation. 1998; 98(Suppl):1-612). Insulin increases the glutathione synthesis by activating gamma-glutamyl-cysteine synthetase. By its metabolic effects, it reduces both polymorphonuclear neutrophils adhesion to ROS (reactive oxygen species—can be effective in post perfusion adhesion of white blood cells to ROS with resultant cellular damage) and stimulated tyrosine phosphorylation.

Reactive oxygen species (ROS) are reactive molecules that contain the oxygen atom that include oxygen ions and peroxides and can be either inorganic or organic. They are highly reactive due to the presence of unpaired valence shell electrons. Cells are able to defend themselves against ROS damage through the use of superoxide dismutases, catalases, lactoperoxidases, glutathione peroxidases and peroxiredoxins. Small molecule antioxidants such as ascorbic acid (vitamin C), tocopherol (vitamin E), uric acid, polyphenol antioxidants, and glutathione also play important roles as cellular antioxidants.

The most important plasma antioxidant in humans is probably uric acid. H₂O₂ induced lipid peroxidation was greatly inhibited by insulin pretreatment. Insulin increased redox status by increasing intracellular glutathione (GSH) content in oxidized cells. These results show that GSH can reverse the effect of oxidation (oxidative free radical damage) on tyrosine kinase activation and phosphorylation, and thus plays an important role in cell signaling, which confirm the antioxidant activity to insulin This is an indication that insulin plays a profound role in maintaining homeostasis, improve cellular physiological function in addition it augments-amplifies the effects of therapeutic agents when locally used as described in this invention at localized tissue levels, in small or large organs like cornea, eye ball, and heart during and after surgery. The GIK is continued to be used in medical practice in cardiac and open heart surgical centers even today. Hence our invention of local use of insulin is very effective in treating dry eye syndrome and related afflictions in similar way.

U.S. Pat. No. 2,145,869 by Dr. Donato Perez Garcia disclose a method for the treatment of syphilis in general and neurosyphilis in particular using subcutaneous insulin injections to induce hypoglycemic shock. Then administer intravenously arsenic, mercury and bismuth, therapeutic agents with glucose and calcium chloride resulting in increased crossing of the blood brain barrier by therapeutic agents to act against the spirochete which causes the neurosyphilis. He described in the invention that the insulin and its hypoglycemic effects increases the antioxidant effect of the therapeutic agents and enhances the permeability of cells. Using this insulin induced hypoglycemic methods; he treated many different kinds of diseases besides neurosyphilis all his life.

U.S. Pat. No. 4,196,196 discloses a composition of insulin, glucose and magnesium dipotassium ethylene diamine tetra acetic acid (EDTA) to enhance tissue perfusion and to facilitate a divalent/monovalent cation gradient uptake in and out of the cells. Insulin in the intravenous infusion with glucose enhances the uptake and activity of potassium and magnesium at the extra and intra cellular level. WE have used this method for decades in many surgical and post surgical patients to alter the potassium level in the extracellular fluid (blood) and cells whenever there was low or high levels of potassium in the serum.

U.S. Pat. No. 4,277,465 discloses an adjuvant to potentiate the absorption insulin from digestive tract for treatment of diabetes by using the enamine derivative. They also describe the use of enamine to facilitate the absorption from intestines of P-lactam antibiotics such as penicillin's and cephalosporin's, high molecular substances such as peptides, carbohydrates such as insulin, heparin, and the peptides include insulin, urokinase, and lysozyme and so on.

U.S. Pat No. 4,971,951 and U.S. Pat. No. 5,155,096 discloses Insulin Potentiation Therapy (IPT) for the treatment of virally related diseases such as herpes, AIDS, Gonorrhea, duodenal ulcer, gall stones, epilepsy, schizophrenia, asthma, arthritis, osteomyelitis, herpes, cancers and many other disease conditions using insulin to deliver the drugs inside the cell with less or non-toxic low doses of various therapeutic, pharmaceutical, biochemical and biological agents or compounds, to enhance the uptake of therapeutic agents with increased augmentation-amplification effects of their effect in non toxic doses.

None of these inventions describe the use of insulin and/or IGF-1 locally to treat dry eye syndrome or any other oculopathies or other local disease condition of the other organs as described in this invention. None of these inventors and patents discloses or describes the local (topical) or regional tissue or organ specific use of insulin and/or IGF-I in a restricted area of the tissue or organ to treat disease states as described here in for treating dry eye syndrome.

Besides Aspirin and antibiotics, insulin is the most commonly used therapeutic agent known to the public and professional alike. Insulin is a hormone secreted by beta cells in the islets of Langerhans in the pancreas. It has been used in home by the patient or in the office by the physician to treat diabetes. It can be easily obtained by prescription and used for treating dry eyes syndrome as described in this invention.

Physiologically, the insulin activates and participates in all the metabolic pathways in the normal, disease afflicted cells systemically and locally; can lead to increased DNA, RNA and protein synthesis which result in increased growth by mitosis (Osborne C K, et al. Hormone responsive human breast cancer in long-term tissue culture: effect of insulin. Proc Natl Acad Sci USA. 1976; 73: 4536-4540); enhances the permeability of cell membranes to many therapeutic agents besides glucose, and electrolytes; it helps and facilitates to move the drugs and therapeutic agent molecules from extra cellular fluid (ECF) to intracellular fluid (ICE) meaning from outside the cells to inside the cells as seen in its use in coronary artery bypass graft (CABG) surgery and in our studies of local effects of insulin. It is a fact that the growth hormone is ineffective in the absence of insulin.

Insulin is an anabolic trophic hormone needed for the growth, multiplication, of all cells in the body including the healthy vascular endothelium, neurons in the retina and macula, as well as secretory glands of the eye lids including the lacrimal glands afflicted with Sjogren's syndrome, and the corneal and conjunctival cells which may be metaplasic in dry eyes syndrome as described above. Increased cellular metabolic activity induced by insulin also enhances the uptake and enhances the action of all therapeutic, pharmaceutical, biochemical and biological agents or compounds by the cells and inside the cell including the cells responsible or involved in the dry eyes syndrome.

Insulin enhances their concentration and effectiveness which has disease-curing qualities. Once inside the cells; the insulin augments and amplifies the effects of any and all therapeutic agents including the agent proven and/or approved to treat dry eyes syndrome including Sjogren's syndrome by increasing activity of all secretory glands, and restoring the physiological function, involved in the production of lacrimal and related secretions.

In our decade of studies and medical practice and experimentation; we found, there is not a single disease except hypoglycemia induced by insulin or otherwise, which cannot be treated using Insulin to enhance the effectiveness of the therapeutic, pharmaceutical, biochemical and biological agents or compounds including the treatment of dry eyes syndrome.

In an ingenious vitro studies, it has been conclusively and methodically demonstrated that the Insulin activates and modifies metabolic pathways in MCF-7 human breast cancer cells, and increase the cytotoxic effect of methotrexate up to 10,000 (ten thousand) fold (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228. Richard L. Schilsky and Frederick. S. Ordway. Insulin effects on methotrexate polyglutamate synthesis and enzyme binding in cultured human breast cancer cells. Cancer Chemother Pharmacol (1985) 15: 272-277).

The data suggest that insulin augmentation of MTX polyglutamate synthesis may account for its previously observed ability to enhance MTX Cytotoxicity. Research studies in human breast cancer and my own studies on every kind of cancer and infection in any part of the body have shown that the group treated with insulin plus low dose methotrexate and other anticancer agents (and/or antibiotics for infection, autoimmune diseases treatments, monoclonal antibody treatment etc.) responded better than the patient treated with insulin or chemotherapy alone (Eduardo Lasalvia-Prisco et al. Insulin-induced enhancement of antitumoral response to methotrexate in breast cancer patients. Cancer Chemother Pharmacol (2004) 53: 220-224. Ayre S G, Perez Garcia y Bellon D, Perez Garcia D Jr (1990) Neoadjuvant low-dose chemotherapy with Insulin in breast carcinomas. Eur J Cancer 26:1262-1263; T. R. Shantha presented at Cancun IPT meeting 2nd meeting 2004 and unpublished studies). These observations supports the findings of Alabastor (IBID) that disease or healthy cell sensitivity to the therapeutic and biological agents such as those to be used to treat dry eyes syndrome could be increased (augmentation-amplification effects) many times by using the method described in this invention using insulin and/or IGF-I.

Our study of injecting Insulin followed by anticancer chemotherapeutic agents directly into cancer masses on hundreds of advanced and localized cancers supports these finding also. Using this method, the palpable tumors including enlarged lymph nodes literally melted away. We treated multiple brain cancer patients by directly injecting insulin with mannitol followed by with specific anti tumor Chemo-therapeutic agents with dextrose and heparin directly infused into the internal carotid artery with positive results. Patients lived longer with good quality of life with less side effects to the chemotherapy agents.

U.S. Patent Application Publication Number: 2004/0054130 Al invention relates to compounds which have the ability to potentiate the physiological activity of insulin, and in particular to small peptide compounds or peptidomimetic compound which has the ability to potentiate one or more of the physiological activities of insulin, in which the peptide comprises a basic amino acid, such as lysine, arginine, homolysine, homoarginine or ornithine; neutral aliphatic amino acid, in either the L- or the D-form, such as glycine, leucine, alanine, phenylalanine or isoleucine, homo leucine, norleucine, homonorieucine, cyclohexylalanine, or homocyclohexylalanine; an aromatic amino acid, such as phenylalanine or tyrosine; and is an amino acid or amino acid analogue which has a side chain having or delocalised electrons. These therapeutic agents can be added to the ophthalmic preparations of the insulin to enhance the insulin activity to treat oculopathies.

U.S. Patent Application Publication Number: 2008/0214441 Al discloses a pharmaceutical association or combination comprising a therapeutic effective amount of insulin or insulin analogue, and a therapeutic effective amount of a pharmaceutically acceptable betaine, in which the insulin and the betaine possibly form a chemical entity or complex, and in which the amount of betaine is adapted for controlling the degradation and/or for increasing the duration of action and/or for enhancing the therapeutically effects of the insulin or insulin analogue.

The present invention provides pharmaceutical compositions and methods for the treatment of diabetes mellitus using combination therapy. The compositions relate to a compound selected from one or more of betaines, lipidic betaines, betaine lipids and an anti diabetic agent such insulin. The methods include the administration of the combination of compound of Formula I, preferably glycine betaine (n=I), with antidiabetic agent where the two components are delivered in a simultaneous manner, where the compound selected from one or more of betaines, lipidic betaines, betaine lipids is administered first, followed by the antidiabetic agent, as well as wherein the antidiabetic agent is delivered first followed by the compound selected from one or more of betaines, lipidic betaines, betaine lipids. In the claims, betaine means pharmaceutically acceptable betaine, lipidic betaines, betaine lipids, pharmaceutically acceptable salts thereof and combinations thereof. The invent ion further relates to a pharmaceutical composition comprising insulin and a betaine wherein the betaine is used to enhance the insulin effects and/or durations. Betaines can be added to ophthalmic insulin preparations to prolong its effect in treatment of various oculopathies.

In general, the present invention relates to pharmaceutical compositions, and more particularly, to pharmaceutical compositions for the treatment of diabetes mellitus using combination therapy. Betaines combinations with insulin's extend the half life and augment the efficiency of insulin's, while protecting patients from cardiovascular events. It is a goal of the present invention to provide stable insulin/betaine pharmaceutical combinations and/or dosages forms suitable to meet patients' needs. Such insulin/betaine combinations are suitable for reducing the necessity of repeated administrations when rapidly and for long periods of time controlling blood glucose in a mammal.

We have used insulin locally as a therapeutic agent in chronic non-healing wounds, burns, after draining the hydrocele of the tunica virginals sac in the scrotum, periodontal disease, post surgical wound healing, delayed healing of broken bones; prostate and bladder afflictions, teeth and gum afflictions, eye and ear diseases and many other diseases; which will be reported later.

The present inventors have used insulin mixed injectate to augment the local anesthetic, or narcotic or steroid effects alone or in combination of the selected therapeutic agents introduced into the epidural or subarachnoid space for the treatment of back pain and/or to relieve other kinds of pain due to different etiologies including post operative and cancer pain with excellent rapid, prolonged pain relief (under study).

The present inventors also used insulin locally in intravenous regional anesthesia (Bier Block) for surgical procedure of the limbs, pain, to treat reflex sympathetic dystrophy (RDS) and complex regional pain syndrome (CRPS) mixed with ketamine, insulin and known selected therapeutic agents. Previously, the other methods to treat RSD have documented with partial success with injectates containing lidocaine and solumedrol, bretylium, guanethidine, reserpine, ketorolac and lidocaine, and non-steroidal anti-inflammatory drugs in saline (Neil Roy Connellya, Scott Reubena and Sorin j. Brullb Y. Intravenous Regional Anesthesia with Ketorolac-Lidocaine for the Management of sympathetically-Mediated Pain. Yale journal of Biology and Medicine 68 (1995), pp. 95-99). We had better success using insulin containing injectates with ketamine with above therapeutic agent's solutions and in addition to the injectates which will be reported later. We had better success using insulin with ketamine directly delivered to CNS in curtailing and curing complex regional pain syndromes (CRPS); reflex sympathetic dystrophy (RSD) & causalgia and many neurological diseases.

The word Prolotherapy means. “PROLO” is short for proliferation, because the treatment causes the proliferation (growth, formation) of new ligament tissue (fibroblasts and collagen formation in the weak, stretched or torn ligaments) in areas where it has become weak resulting in pain with movement (Ross A. Hauser, Marion A. Hauser. 2007. Prolo Your Pain Away! Curing Chronic Pain with Prolotherapy. Chicago-Amazon books). Many solutions are used in inducing ligamenotous growth such as, dextrose (10%-25%) with lidocaine (a local anesthetic 0.1-0.2%), phenol, glycerin, cod liver oil extract; solution containing 1.25% phenol, 12.5% dextrose and 12.5% glycerin; Glucose 20% and Lidocaine 0.1% solution; mixture of 1 cc of 5% sodium morrhuate and 1 cc of 1% lidocaine and Dr. DeHaan's “Prolo Cocktail” containing 25% of each of the following substances: 50% dextrose, 2% lidocaine or procaine (without epinephrine), vitamin B12 (1000 mcg/ml), and Biosode (“a homeopathic with growth and Krebs cycle energy factors”) has been used.

The inventors have used glucose along with insulin and lidocaine in our prolotherapy injectate for various musculoskeletal pain, including arthritis, back pain, neck pain, fibromyalgia, sports injuries, unresolved whiplash injuries, carpal tunnel syndrome, chronic tendonitis, partially torn tendons, ligaments and cartilage, degenerated or herniated discs, TMJ pain and sciatica. It is important to note the principle of prolotherapy is to induce fibroblasts to multiply and lay more ligaments (collagen); make the ligaments and tendons stronger inducing sterile inflammation at the site. Insulin can enhance the multiplication of fibroblasts by these prolotherapy and other sterile inflammatory therapeutic agents to make ligaments stronger.

With insulin, the prolotherapy was more effective compared to when the prolotherapy therapeutic agent was used alone. Insulin increased the fibroblast mitosis and thus increased production of collage to strengthen the ligaments of the painful joint giving long lasting fast pain relief with stronger functional joints. When insulin is therapeutically effective ever in taking away the pain by various prolotherapy therapeutic agents, one can see how effective it is in treating the dry eye syndrome and associated diseases of the eye.

Trigger points or trigger sites are described as hyperirritable spots in skeletal muscle that are associated with palpable nodules in taut bands of muscle fibers whose compression may elicit local tenderness, referred pain, or local twitch response. There are many therapies to take away the tenderness and sore spots. Various injections can be used including saline, local anesthetics such as procaine hydrochloride (Novocain); a mixture of lidocaine and marcaine without steroids (steroids can cause muscle damage, hence contraindicated) is used to relive the pain. Trigger pain point injection for myofacial pain, fibromyalgia, tennis elbow, intercostals pain, wrist and back pains, and injection of joints with therapeutic agents such as local anesthetic with insulin resulted in rapid and effective relief of pain compared to injectate without insulin.

The same methods can be used to treat the dry eyes syndrome, Sjogren's syndrome, and any condition contributing to the dry eye syndrome alone, and all painful conditions of the eye in combination with other known therapeutic, pharmaceutical, biochemical and biological agents or compounds as described above.

In an important experiment, Zheng et al showed the role of insulin like growth factor-I (IGF-I) which have insulin like effects; induced the inner ear epithelial cell culture growth (Zheng, J. L., Helbig, C. & Gao, W-Q. Induction of cell proliferation by fibroblast and insulin-like growth factors in pure rat inner ear epithelial cell cultures. J. Neurosci. 17:216-226 (1997). There is a clear indication that insulin and IGF-I not only play a role in potentiation of (augmentation-amplification effects) the therapeutic, pharmaceutical, biochemical and biological agents or compounds; they can independently stimulate cells growth in eye structures including various glands that produce tears. (Shantha T. R., Unknown Health Risks of Inhaled Insulin. Life Extension, September 2007 pages 74-79, Post publication comments in September 2008 issue of Life Extension, Pages 24. Shantha T. R and Jessica G. Inhalation Insulin, Oral and Nasal Insulin Sprays for Diabetics: Panacea or Evolving Future Health Disaster. Part I: Townsend Letter Journal: Issue #305, December 2008 pages: 94-98; Part II: Townsend Letter, January 2009, Issue #306, pages-106-110).

The normal cell undergoes the following changes as pathological state takes its root: 1. Dysplasia, in which cell maturation and differentiation are delayed, often indicative of an early neoplastic process. The term dysplasia is typically used when the cellular abnormality is restricted to the originating tissue, as in the case of an early, in-situ neoplasm. It means that the original cells are not robust enough to withstand the new environment, and so they change into another type more suited to the new environment.

2. Metaplasia is the reversible replacement of one differentiated cell type with another mature differentiated cell type. The medical significance of metaplasia is that in some sites cells may progress from metaplasia, to develop dysplasia, and then malignant neoplasia (cancer).

3. It is also contrasted with heteroplasia, which is the abnormal growth of cytological and histological elements without a stimulus. Insulin has profound effect on these cells undergoing metaplasia, dysplasia, and heteroplasia as indicated in our above articles published in life Extension and Townsend letters research publications; so also on the glandular cells of ocular pathology contributing eye diseases including dry eye syndrome especially when therapeutic agents are combined with insulin.

Insulin exert its trophic effect on the cell physiology without discriminating whether it is normal, metaplasic, dysplasic, heteroplasic or carcinogenic (Philpott M P, Sanders D A, Kealey T. Effects of insulin and insulin-like growth factors on cultured human hair follicles: IGF-I at physiologic. J Invest Dermatol 1994; 102: 857-61, Shantha IBID). It is a known physiological phenomenon that the insulin does bind to the receptor sites of the IGF-I and insulin, and exert multiple profound physiological, pharmacological therapeutic effects and induce cell growth (besides glucose transport), such as enhanced metabolism, enhances mitosis, enhances (augmentation-amplification effects) the therapeutic effect of other pharmacological agents as reported (Shantha T. R., Life extension September 2007: 74-79,) on the cell to which it binds has been reported in above publications.

Thus any dysfunction of eyelids and lacrimal glands; damage to the corneal and conjunctiva seen in many cases of dry eye syndrome will be restored back to normal using the described inventive method. None of the present eye drops for the dry eye syndrome contain therapeutic agents to repair and restore the damaged or disease afflicted cells and tissues involved in hydration and coating of the exposed cornea and conjunctiva.

Insulin, potassium and glucose are routinely administered to treat low potassium levels in the cells even to this day. The inventor has used this method to lower the potassium levels in the blood for 3 decades. Insulin and glucose facilitates the entry of potassium inside the cell—a life saving measure. Similarly, the Insulin deposited in the conjunctival sac will enhance the uptake of therapeutic, pharmaceutical, biochemical and biological agents or compounds by the dysfunctional cells as described in this inventive method.

Inventors have used insulin as potentiator of uptake and enhancer of therapeutic action of diverse therapeutic agents to cure and/or curtail curable acute, chronic and incurable diseases such as cancer, Lyme disease, scleroderma, lupus, psoriasis; antibiotic resistant staphylococcus infection, MRSA infection, chronic wounds, neurological diseases, inner and middle ear affliction, autoimmune diseases, leprosy, prostate pathologies, skin diseases, herpes zoster of the eye with antiviral agents, tuberculosis, and many other diseases with good results.

Inventors' also have used insulin with other specific treatment modalities against depression, Alzheimer's, Autism, Parkinson's and many other neurological diseases successfully. It needs to be delivered to the brain through proper routes which are going to report in later publications and as described in our provisional patent on rabies cure presentations (Shantha, T. R. Site Of Entry Of Rabies Virus Form The Nose And Oral Cavity; And New Method Of Treatment Using Olfactory Mucosa And By Breaking BBB, presented at The 2^{nd} International Rabies In Asia Conference Held In Hanoi, 2009, Pp 70-73, and The Rabies in the North Americus (XX RITA), held in Quebec City, 2009, Pp 20-21, Rabies cure, patent pending 2009).

The present inventors have used insulin for more than a decade to enhance the effectiveness of locally injected therapeutic agents especially cancers with chemotherapeutic agents with remarkable results. Our data also supports that the insulin spray on indolent ulcers anywhere in the body, including the oral (gums) and nasal cavity augmented the healing. It stimulated the fibroblast, endothelial cell, angiogenesis and skin cell growth resulting accelerated wound healing. Application of insulin soaked cotton swabs (1-3 units in normal saline) after teeth extraction induces rapid healing with reduced pain. Studies show that the application of insulin and antibiotics locally on the gums eliminated gum diseases (periodontitis), made the loose teeth firm, cleared the root infection rapidly which is under study (Dr. Hughes, J. DDS: Personal communication).

Insulin is a metabolic activity enhancer of all cells and therapeutic agents. Hence it can play an important role in treatment many diseases including dry eyes syndrome by increasing the metabolic activity of tear producing glandular system (Shantha T. R.; 1. discovery of insulin and IPT: amazing history, 2. high dose methotrexate therapy using Insulin; 3 local injections of tumors with insulin and cytotoxic drugs; 4. two and three cycle insulin Potentiation therapy: Presented at 2^{nd} international conference on Insulin Potentiation Therapy held at Cancun, Mexico, Jun. 28-Jul. 1, 2004).

A synergy between certain membrane and metabolic effects of insulin on cell molecular biology increases therapeutic efficacy of all anti dry eyes syndrome therapeutic, pharmaceutical, biochemical and biological agents or compounds and it does so with reduced doses of the drugs, enhancing their uptake with augmentation-amplification effects greater than before therapeutic efficacy once it enters the cells, increasing the safety of therapeutic agents. Thus the present inventive method not only enhances the uptake of therapeutic agents, but also enhances their therapeutic effect inside the cells of the disease afflicted cells.

It is known that the pharmaceutically acceptable oxidizing agent facilitates the delivery of the bioactive agent through the skin and mucous membranes such as oral cavity, nasal passages and conjunctiva. In general, the oxidizing agent can react with molecules present in the conjunctiva would adversely react with the bioactive agent. For example, reduced glutathione present in the mucus membranes of the eyes and skin can inactivate bioactive agents such as insulin by breaking chemical molecular bonds. Not wishing to be bound by theory, when delivering insulin through the skin and conjunctiva, reduced glutathione can inactivate insulin.

Specifically, insulin has numerous disulfide bonds which are crucial for its protein conformation, biological activity, and subsequent therapeutic effects. Reduced glutathione will inactivate insulin by reducing or breaking insulin's disulfide bonds. Once these disulfide bonds are broken; insulin becomes inactive due to lost protein conformation and biological activity. Thus, the administration of the oxidant by eye drops (as described by Shantha et al in U.S. Patent Application Pub. No. 2009/0347776 A1) herein prevents the inactivation of the bioactive agent such as insulin when applied to the skin and conjunctival sac of the eye.

Specifically, applying an oxidant or a pharmaceutically oxidizing agent to conjunctival sac will lower or prevent the effects reduced proteins and reduced biological molecules have on the bioactive agents. In this manner, the inactivation of bioactive agents via reduction or cleavage of crucial molecular bonds will be avoided. The selection and amount of the pharmaceutically acceptable oxidizing agent can vary depending upon the bioactive agent that is to be administered. In one aspect, the oxidizing agent includes, but is not limited to, iodine, povidone-iodine, and any source of iodine or combinations of oxidants, silver protein, active oxygen, potassium permanganate, hydrogen peroxide, sulfonamides, dimethyl sulfoxide or any combination thereof.

These oxidizing agents may also act as absorption agents which help facilitate delivery of a therapeutic agent onto and into a skin. In one aspect, the oxidant is at least greater than 1% weight per volume, weight per weight, or mole percent. In another aspect, the mucosal membrane permeability enhancer may be at least greater than 1.5%. 2.0%. 20 2.5%, 3.0%, 3,570, 4.0%, or 4.5% weight per volume, weight per weight, or mole percent. In this aspect, the oxidant may range from 2% to 10%, 2% to 9.5%. 3% to 8%, 3% to 7%. or 4% to 6% weight per volume, weight per weight, or by mole percent.

Our preliminary studies have shown that the conjunctiva unlike normal skin may not act as a barrier for entry of insulin due to the paucity of the presence of reduced glutathione. It is likely that conjunctiva hardly contains any insulin blocking agent, besides; it does not have the multilayered stratum coneium as seen on the skin which blocks the entry of insulin from the skin. The insulin deposited in the conjunctival sac is rapidly absorbed and reaches the lining cells of the lids and its glandular system, cornea and bulbar conjunctiva, and helps all secretory glands including lacrimal glad to recover from any pathological state affecting their secretory function; restore the coating of the cornea and sclera and prevents the dry eyes syndrome.

In one aspect, transconjunctival penetration of insulin and therapeutic, pharmaceutical, biochemical and biological agents or compounds can be facilitated by enhancers that can be used to further expedite the entry of these agents into the glandular system of the eye lids and lacrimal gland. Penetration enhancers not only penetrate a membrane efficiently, but these enhancers also enable other bioactive agents to cross a particular membrane more efficiently. Penetration enhancers produce their effect by various modalities such as disrupting the cellular layers of the conjunctival sac surface interacting with intracellular proteins and lipids, or improving partitioning of bioactive agents as they come into contact with the mucosal membranes.

With these enhancers, macromolecules up to 10 kDa are able to pass through the conjunctival sac layers of the eyes reaching the site of dry eyes syndrome where the blood vessels and retina are undergoing pathological changes. These enhancers should be non-toxic, pharmacologically inert, non-allergic substances. In general, these enhancers may include anionic surfactants, urea's, fatty acids, fatty alcohols, terpenes, cationic surfactants, nonionic surfactants, zwitterionic surfactants, polyols, amides, lactam, acetone, alcohols, and sugars. In one aspect, the 10 penetration enhancer includes dialkyl sulfoxides such as dimethyl sulfoxide (DMSO),decyl methyl sulfoxide, dodecyl dimethyl phosphine oxide, octyl methyl sulfoxide, nonyl methyl sulfoxide, undecyl methyl sulfoxide, sodium dodecyl sulfate and phenyl piperazine, or any combination thereof. In another aspect, the penetration enhancer may include lauryl alcohol, diisopropyl sebacate, oleyl alcohol, diethyl sebacate. dioctyl sebacate, dioctyl azelate, hexyl laurate, ethyl caprate, butyl stearate, dibutyl sebacate, dioctyl adipate, propylene glycol dipelargonate, ethyl laurate, butyl laurate, ethyl myristate, butyl myristate, isopropyl palmitate, isopropyl isostearate, 2-ethylhexyl pelargonate, butyl benzoate, benzyl benzoate, benzyl salicylate, dibutyl phthalate, or any combination thereof.

In one aspect, the skin permeability enhancer is at least greater than 1% weight per volume, weight per weight, or mole percent. In another aspect, the mucosal membrane permeability enhancer may be at least greater than 1.5%, 2.0%, 2.5%, 3.0%. 3.5%. 4.0%. 4.5% up to 50% weight per volume, weight per weight, or mole percent. In one aspect, the mucosal membrane permeability enhancer is dimethyl sulfoxide. In this aspect, the amount of dimethyl sulfoxide may range from 2% to 10%. 2% to 9.5%. 3% to 8%. 3% to 7% or 4% to 6% weight per volume, weight per weight, by mole percent, or any effective therapeutic amount.

In other aspects, these additional components may include antiseptics, antibiotics, anti-vitals, anti-fungals, anti-inflammatories, anti-dolorosa, antihistamines, steroids, vasodilators and/or vasoconstrictors to reduce inflammation, irritation, or reduce rapid absorption through conjunctival sac. Such vasoconstrictors may include phenylephrine, ephedrine sulfate, epinephrine, naphazoline, neosynephrine, vasoxyl, oxyrnetazoline, or any 5 combinations thereof. Such anti-inflammatories may include non-steroidal anti-inflammatory drugs (NSAIDs). NSAIDs alleviate pain and inflammation by counteracting Cyclooxygenase and preventing the synthesis of prostaglandins. In one aspect, NSAIDs include celecoxib, meloxicam, nabumetone, piroxicam, napmxen, oxaprozin, rofecoxib, sulindac, ketoprofen, valdewxid, anti-tumor necrosis factors, 10 anti-cytokines, anti-inflammatory pain causing bradykinins or any combination, thereof.

Such antiseptics, anti-virals, anti-fungals, and antibiotics, may include ethanol, propanol, isopropanol, or any combination thereof; a quaternary ammonium compounds including, but not limited to, benzalkonium chloride, cetyltrimethylammonium bromide, cetylpyridinium chloride, benzethonium chloride, or any combination thereof; boric acid; chlorhexidine gluconate, hydrogen peroxide, iodine, mercurochrome, ocetnidine dihydrochloride, sodium chloride, sodium hypochlorite, silver nitrate, colloidal silver, mupirocin, erthromycin, clindamycin, gentamicin, polymyxin, bacitracin, silver, sulfadiazine, or any combination thereof. It is intent of this invention to use of the insulin along with above described anti-inflammatory antibacterial agents that can eliminate the pathogenic factors contributing to the dry eyes syndrome and restore normal sight.

In accordance with one aspect of the invention, the compounds used to apply locally to the eye lids site are mixed with a conjunctivally well-suited vehicle or carrier. The eye lid applications can be easily done in cases of Meibomian gland dysfunction (MGD). The compositions of this invention may comprise aqueous solutions such as e.g., physiological saline, oil, gels, patches, solutions or ointments. The vehicle which carry these biologically active therapeutic agents may contain conjunctivally compatible preservatives such as e.g., benzalkonium chloride, surfactants like e.g., polysorbate 80, liposome's or polymers, for example, methyl cellulose, polyvinyl alcohol, polyvinyl pyrrolidone and hyaluronic acid etc. Sterile water or normal saline are used in some of the preparations of the eye drops in this invention.

There are various forms of insulin used to treat diabetes which can be formulated to be used in this invention. They are grouped under rapid, short, intermediate, and long acting insulin. It is also dispensed as premixed form containing rapid to long acting insulin. Insulin products are categorized according to their putative action (see Table IV) profiles as:

1. Rapid-acting: insulin lispro, insulin aspart, and insulin glulisine

2. Short-acting: regular (soluble) insulin

3. Intermediate-acting: NPH (isophane) insulin

4. Long-acting: insulin glargine and insulin detemir

The table IV summarizes the time of onset; peak action and duration of action of different type and brands of insulin that can be used in our invention.

| | | | | |
|---|---|---|---|---|
| Insulin Preparation | Onset of | Peak | Effective | Maximum |
| and their gerneric and | Action in | action in | duration of | duration |
| trade names | hours | hours (h) | action (h) | in hours |

RAPID - ACTING INSULIN ANALOGUES AND PREPARATIONS

| | | | | |
|---|---|---|---|---|
| Insulin lispro | ¼-½ | ½-1¼ | 3-4 | 4-6 |
| (Humalog), Insulin | | | | |
| aspart (NovoLog), | | | | |
| Insulin glulisine | | | | |
| (Apidra) | | | | |

SHORT - ACTING INSULIN

| | | | | |
|---|---|---|---|---|
| Regular (soluble) | ½-1 | 2-3 | 3-6 | 6-8 |

INTERMEDIATE-ACTING

| | | | | |
|---|---|---|---|---|
| NPH (isophane) | 2-4 | 6-10 | 10-16 | 14-18 |

LONG - ACTING INSULIN ANALOGUES

| | | | | |
|---|---|---|---|---|
| Insulin glargine | 3-4 | 8-16 | 18-20 | 20-24 |
| (Lantus) | | | | |
| Insulin detemir | 3-4 | 6-8 | 14 | ~20 |
| (Levemir) | | | | |

Preparation of the Dry Eye Syndrome Patients for Therapy Using Our Inventive Method Using Insulin

Before using described inventive methods and examples; a thorough examination of the affected patients eye is in order. The examination of the eye may include: 1. Acuity testing, 2. Biomicroscopy, 3. Intraocular pressure (IOP), 4. Ophthalmoscopy, 5. Color vision test, 6. Tear osmolality, 7. Schimer's test, 8. Tear film breakup time (tBUT), 9. Superficial punctate keratitis (SPK), 10. Fluorescein and Rose Bengal staining (RBS) 11, slit-lamp examination of the conjunctiva, cornea, anterior chamber, iris, and lens, 12. The Ocular Surface Disease Index (OSDI), 13. Microscopic examination of the tear filament, 14. Maturation index (a Papanicolaous stained sample of conjunctival epithelium). In addition, a complete physical examination with blood testing for FSH, LH, estradiol and testosterone levels and a urine sample for pregnancy test when deemed necessary.

To apply eye drops of the therapeutic agents, wash hands with mild antiseptic soap. Be careful not to touch the dropper tip or let it touch your eye lids to avoid contamination if there is eye lid infection. Tilt the head back, or lay down with head extended on a neck pillow; gaze upward and backwards; and pull down the lower eyelid to expose the conjunctival fornix. Place dropper directly over eye away from the cornea and instill the prescribed number of drops. Look downward and gently close your eye for 1 to 2 minutes. Try not to blink and do not rub the eye. Do not rinse the dropper unless one knows how to sterilize in hot water.

If other therapeutic, pharmaceutical, biochemical and biological agents or compounds are to be selected to treat the condition with our invention; wait at least 3 minutes before using other selected anti-dry eyes syndrome therapeutic agents or the other kinds of ophthalmic medicaments. It is important to instill medications regularly and exactly as prescribed to control dry eyes syndrome. Consult your doctor and/or pharmacist if the systemic medications you are taking together are safe to use with eye drops described. There is no contraindication for insulin eye drop use except those with hypoglycemia syndromes and external ocular tumors.

To minimize absorption into the bloodstream and maximize the amount of drug absorbed in the eye, close your eye for one to five minutes after administering the drops and press your index finger lightly against the inferior nasal corner of your eyelid to close the tear duct which drains into the nose (FIG. 4). This will prevent any adverse systemic effects due to nasal vascular uptake to systemic circulation from the nasolacrimal duct drainage of the therapeutic agents from the conjunctival sac. Eye drops may cause an uncomfortable burning or light stinging sensation which should last for only a few seconds. The anti-dry eyes syndrome drops take effect after 5 to 10 minutes after application depending upon the therapeutic agents used with it. This process can be repeated every 6-12 or 24 hours for 3-7 days a week till the desirable results are obtained. The therapeutic agents are instilled using sterile dropper (or bottle with medication equipped with a dropper nipple) into the conjunctival sac.

Experiments by the present inventors has shown that the local application of rapid action or other types of insulin formulations on the balding scalp, eye lid hair line, on the gums, oral and nasal mucosa, and conjunctival sac, surgical wounds, raw area of extracted wisdom teeth, local injections of tumors, injection into tunica vaginalis testes, and other regional and local site did not change the blood sugar levels (no hypoglycemic effects) indicating that it is safe to use up to 1-2-3 IU insulin to the conjunctival sac of both eyes without hypoglycemia effects. The present invention formulation contains only 0.05 IU per drop which can be increased in severe disease states.

The typical threshold for hypoglycemia is 70 mg/dL (blood sugar level of 3.9 mmol/L), although it may be higher or lower depending on a patient's individual blood glucose target range. Generally, the hypoglycemia is defined as a serum glucose level (the amount of sugar or glucose in a persons blood) below 70 mg/dL. Symptoms of hypoglycemia typically appear at levels below 60 mg/dL. Some people may feel symptoms above this level. Levels below 50 mg/dL affect of brain function. Signs and Symptoms of hypoglycemia include erratic or rapid heartbeat, sweating, dizziness, confusion, unexplained fatigue, shakiness, hunger, feeling of heat, difficulty in thinking, confusion, headache, seizures, and potential loss of consciousness if severe hypoglycemia develops. Once symptoms of hypoglycemia develop, it should be treated immediately with oral ingestion of a fast-acting carbohydrate such as glucose tablets, fruit juice, fruit bowl, chocolate bar, or regular Coca-Cola, sugary drinks or eat plain sugar followed with a drink of water.

Preparation of Insulin Eye Drops for Use in Dry Eye Syndrome

Take 100 international units (IU) of rapid or intermediate or long acting insulin (or IGF-1) and dilute in 10 ml of sterile saline or distilled water or other carriers and facilitators as described above. The pH can be adjusted to prevent the sting when dropped to the conjunctival sac and can also add nanograms (micrograms) of local anesthetics also to prevent the stinging. In this preparation each ml contains 10 units of insulin. In pharmacies, a drop used to be another name for a minimum, which would make it 0.0616 milliliters. But now the drop is standardized in the metric system to equal exactly 0.05 milliliters. That is 20 drops make one ml (1 cc). That means each drop contains 0.05 IU of insulin. The concentration of the insulin content can be increased to 0.075, 0.10, 0.20, and 0.05, 0.10 IU or more of insulin per drop by increasing the insulin content in the dilutant preparation. It can be also decreased by reducing the insulin units used for the preparation of the ophthalmic drops. Instill one to two drop to each eye lower lid fornix and/or everted upper eyelid (conjunctival sac) as a single agent.

If other combination anti-dry eyes syndrome therapeutic agent is to be used first use insulin drops, wait for 3-5-10 minutes and apply the other therapeutic, pharmaceutical, biochemical and biological agents or compounds. After this procedure, instill one more insulin drop to further enhance the uptake of the other selected therapeutic agents to augment-amplify their effects at cellular level. This step is optional and may not be needed in most of the cases. The dose used in our invention can be appropriately selected depending upon symptom, age, dosage form, existing health conditions etc. The pH can be within a range which is acceptable to ophthalmic preparations and, preferably within a range from 4 to 8.

The data we have supports that if the other therapeutic agents are used after insulin, they be prepared in 5-10% solutions of glucose. The glucose acts as a carrier of the therapeutic agents after pretreatment with insulin, which I have named local Insulin Potentiation Therapy (Local IPT).

Insulin can be compounded as a liquid ophthalmic isotonic solution containing cyclosporin, or other antiautoimmune therapy agents such as methotrexate or cyclophosphamide, or vitamins, and one or more one buffering agents, said buffering agents producing a pH in said composition similar to mammalian eye fluids.

The insulin pharmaceutical eye drop preparation of this invention may contain 0.25%-0.5%-1%-2% or more glucose. There are several mechanisms by which glucose and insulin protect the damaged cells and restores normal function. Glucose is the preferred substrate during periods of cell damage and ischemia. Adenosine triphosphate derived from glycolysis is vital for stabilization of membrane ion transport, which in turn is crucial to the glandular cellular integrity, endothelium, vascular smooth muscle cells, nerve fibers and their terminals. Preservation of these function in the secretory glands of the eye, cornea, and conjunctival lining decrease any further damage and participates in their repair.

Glucose also esterifies intracellular free fatty acids, which decreases their toxic end-products, oxygen free radicals. Because glucose is a direct precursor of pyruvate, which is carboxylated to the citric acid cycle substrates malate and oxaloacetate, it can replenish depleted substrates, thus stimulating oxidative aerobic metabolism. Glucose with the help of insulin also esterifies intracellular free fatty acids, which decreases their toxic end-products, oxygen free radicals.

Experimental studies have shown that glucose converted to pyruvate with the help of insulin can restore secretory function through the replenishment of depleted citric acid substrates thus helping in repair and restoring the glandular cellular function and helping in curtailing or curing the dry eye syndrome and Sjogren's syndrome. Experimental studies have also shown that the glucose is converted to pyruvate in the presence of insulin that can restore contractile function through the replenishment of depleted citric acid. Thus our invention of insulin with glucose can make smooth muscle cells around the secretory glands and ducts contract with a stimulus and pour their secretions to conjunctival sack relieving the dry eye syndrome.

Insulin stimulates pyruvate dehydrogenase activity, which in turn stimulates aerobic metabolism. Exogenous insulin helps to reverse insulin resistance during which can be of benefit in dry eye syndrome associated with diabetes. It is also important to note that the glucose with insulin facilitates the entry of other therapeutic agents into the normal and disease afflicted cells.

The above pharmaceutical eye drop preparation of our invention may contain antibacterial components which are non-injurious to the eye when used. Examples are: thimerosal, benzalkonium chloride, methyl and propyl paraben, benzyldodecinium bromide, benzyl alcohol, or phenylethanol. Due to autism controversy, we will avoid using thimerosal.

The therapeutic pharmaceutical preparation may also contain buffering ingredients such as sodium chloride, sodium acetate, gluconate buffers, phosphates, bicarbonate, citrate, borate, ACES, BES, BICINE, BIS-Tris, BIS-Tris Propane, HEPES, HEPPS, imidazole, MES, MOPS, PIPES, TAPS, TES, and Tricine.

The therapeutic, pharmaceutical, biochemical and biological agents or compounds used in our invention may also contain a non-noxious pharmaceutical carrier, or with a non-toxic pharmaceutical inorganic substance. Typical of pharmaceutically acceptable carriers are, for example, water, mixtures of water and water-miscible solvents such as lower alkanols or aralkanols, vegetable oils, peanut oil, polyalkylene glycols, petroleum based jelly, ethyl cellulose, ethyl oleate, carboxymethyl-cellulose, olyvinylpyrrolidone, isopropyl myristate and other traditionally acceptable carriers.

The therapeutic preparation may also contain non-toxic emulsifying, preserving, wetting agents, bodying agents, as for example, polyethylene glycols 200, 300, 400 and 600, carbowaxes 1,000, 1,500, 4,000, 6,000 and 10,000, antibacterial components such as quaternary ammonium compounds, phenylmercuric salts known to have cold sterilizing properties and which are non-injurious in use, methyl and propyl paraben, benzyl alcohol, phenyl ethanol, buffering ingredients such as sodium borate, sodium acetates, gluconate buffers, and other conventional ingredients such as sorbitan monolaurate, triethanolamine, oleate, polyoxyethylene sorbitan monopalmitylate, dioctyl sodium sulfosuccinate, monothioglycerol, thiosorbitol, ethylenediamine tetracetic. Furthermore, appropriate ophthalmic vehicles can be used as carrier media for the current purpose including conventional phosphate buffer vehicle systems, isotonic boric acid vehicles, isotonic sodium chloride vehicles, isotonic sodium borate vehicles and the like.

The objects are accomplished by treating the eye with an aqueous composition containing an effective amount of a nonionic surfactant and insulin. Applicant has found that an effective amount of surfactant may comprise anywhere from 0.5 percent by weight and volume to about 10 percent by weight and volume (hereinafter %), preferably about 1-5%, of active surfactant (not combined with oil) in the composition combined with insulin. However, it should be noted that the use of any oil in the composition will reduce the effectiveness of the surfactant. The reason is that a substantial percentage of the surfactant tends to serve as a vehicle for dissolving or forming an emulsion of the oil with the aqueous layer to “wash” or hydrate the corneal surface. Thus, if any oil is used in the composition, then additional surfactant will be required to provide the effective amount of 0.5-10% preferably 1-5%, of available active nonionic surfactant.

The anti-dry eyes syndrome therapeutic agents preparation may also contain surfactants such as polysorbate surfactants, polyoxyethylene surfactants (BASF Cremaphor), phosphonates, saponins and polyethoxylated castor oils, but preferably the polyethoxylated castor oils which are commercially available.

The pharmaceutical preparation may too contain wetting agents that are already in use in ophthalmic solutions such as carboxymethylcellulose, hydroxypropyl methylcellulose, glycerin, mannitol, polyvinyl alcohol or hydroxyethylcellulose and the diluting agent may be water, distilled water, sterile water, or artificial tears. The wetting agent is present in an amount of about 0.001% to about 10%.

The ophthalmic formulation of this invention may include acids and bases to adjust the pH; tonicity imparting agents such as sorbitol, glycerin and dextrose; other viscosity imparting agents such as sodium carboxymethylcellulose, polyvinylpyrrolidone, polyvinyl alcohol and other gums; suitable absorption enhancers, such as surfactants, bile acids; stabilizing agents such as antioxidants, like bisulfites and ascorbates; metal chelating agents, such as sodium EDTA; and drug solubility enhancers, such as polyethylene glycols. These additional ingredients help make commercial solutions with stability so that they need not be compounded as needed.

Ophthalmic medications compositions will be formulated so as to be compatible with the eye and/or contact lenses. The eye drop preparation should be isotonic with blood. As will be the ophthalmic compositions intended for direct application to the eye will be formulated so as to have a pH and tonicity which are compatible with the eye. This will normally require a buffer to maintain the pH of the composition at or near physiologic pH (i.e., pH 7.4) and may require a tonicity agent to bring the osmolality of the composition to a level at or near 210-320 millimoles per kilogram.

In the following detailed description of the invention, reference is made to the drawings, microphotographs and tables in which reference numerals refer to like elements, and which are intended to show by way of illustration specific embodiments in which the invention we describe using insulin, and IGF-1 with or without other known anti dry eyes syndrome therapeutic, pharmaceutical, biochemical and biological agents or compounds enumerated here may be prescribed and practiced. It is understood that other embodiments may be utilized and that structural changes may be made without departing from the scope and spirit of the invention described herein.

The eye drop composition of the invention includes buffering agents to adjust the acidity or alkalinity of the final preparation to prevent eye irritation. The composition is an isotonic solution in that it have the similar pH to as fluids indicating that that the pH of the composition be 6.1. to 6.3 or 7.4. The buffering agents may include all of zinc sulfate, boric acid and potassium necessary to be effective in achieving a pH of the composition of from 6.1 to 6.3. or up to 8. Typically, the total amount of buffering agents present in the composition ranges from 1 to 10% by weight of the composition.

The eye drop composition also, if possible, includes a lubricant such as cellulose derivatives (carboxymethyl cellulose). The composition may also contain known preservatives conventionally used in eye drop such as benzalkonium chloride and other quaternary ammonium preservative agents, phenylmercuric salts, sorbic acid, chlorobutanol, disodium edetate, thimerosal, methyl and propyl paraben, benzyl alcohol, and phenyl ethanol. Purified benzyl alcohol may be in the concentration preferably from 0.1 to 5% by weight.

The eye treatment composition of the invention is a solution first having a vehicle of water or mixtures of water and water-miscible solvents such as, for example, lower alkanols or arylalkanols, phosphate buffer vehicle systems, isotonic vehicles such as boric acid, sodium chloride, sodium citrate, sodium acetate and the like, vegetable oils, polyalkylene glycols, and petroleum based jelly, as well as aqueous solutions containing ethyl cellulose, carboxymethyl cellulose and derivatives thereof, hydroxypropylmethyl cellulose, hydroxyethyl cellulose, carbopol, polyvinyl alcohol, polyvinyl pyrrolidone, isopropyl myristate and other conventionally-employed non-toxic, pharmaceutically acceptable organic and inorganic carriers.

Because the composition is applied to the eye, the composition should be sterile in the form of an isotonic solution. The constitution may also contain non-toxic supplementary substances such as emulsifying agents, wetting agents, bodying agents and the like such as, for example, polyethylene glycols, carbowaxes, and polysorbate 80. Other conventional ingredients can be employed such as sorbitan monolaurate, triethanolamnine, oleate, polyoxyetbylene sorbitan 35 monopalmitylate, dioctyl sodium sulfosuccinate, monothioglycerol, thiosorbitol, ethylenediamine tetraacetic acid, and the like.

The Following are the Examples of Using Our Invention Insulin and/or IGF-1 Biological Factors Alone or in Combination with Known Therapeutic, Pharmaceutical, Biochemical and Biological Agents or Compounds to Treat Dry Eye Syndrome

### EXAMPLE 1

Select the patient and establish the type of dry eye syndrome and its etiology the person is suffering. Test both the quantity and the quality of tear by measuring tear production using the Schirmer tear test (optional). Record the degree of corneal and conjunctival damage as a result of dry eye syndrome by using fluorescein or Rose Bengal staining agents instilled into conjunctival sac followed by the thorough examination of the cornea and the ocular surfaces using the magnification of a slit-lamp utilizing filters to intensify the natural fluorescence of these dyes after one minute after application of the stains. The damage to the tissue if there is any is revealed as “staining”, which is the infiltration of the dye into the cell or between the tight junctions of the cells.

Position the patient in supine posture or standing with head extended on a support or hyper extended. Using a dropper, or dropper bottle containing the insulin formulations are instilled one to two drop of insulin preparation in each eye lower lid fornix and/or everted upper eyelid. Apply slight pressure at the nasal angle of eye pressing on the nasolacrimal canaliculi-sac-duct to prevent leaking of the therapeutic agents to the nose to avoid systemic absorption and its adverse effects as shown in the FIG. 4. Stay still for 2-3-5 minutes and resume the desired posture. These instructions should be given to the patients and trained how to use the dry eye syndrome eye drops appropriately.

### EXAMPLE 2

A topical emulsion of cyclosporin for treating KCS has been FDA approved and promoted under the trade name Restasis™ (Allergan, Inc., Irvine, Calif.). It is a mixture of cyclosporin combined with a higher fatty acid glyceride, such as castor oil, and a surface active agent, such as polysorbate 80, and an emulsion stabilizer, such as a cross-linked polyacrylate. It acts by decreasing the inflammation on the eye surface (probably tear glandular system) and helps to increase the production of healthy tears. However, treatment with an emulsion containing oily droplets can result in eye irritation or a clouding of visual field. Due to oily preparation, the active ingredient is less bioavailable. Restasis is not appropriate for immediate relief for an uncomfortable irritated eye as it may take up to 6 months for maximum improvement (source: The Eye Digest). To make more effective, add insulin to the preparation so that Insulin can enhance the uptake of cyclosporin, and augment-amplify the effects of the cyclosporins in it. Due to this biological effect, less cyclosporin can be added in the final cyclosporin preparation; at the same time less time is needed inside the afflicted cells to achieve the desired effects. Using insulin before or with it will enhance the activity of Restasis. With insulin, Restasis will become more effective within weeks instead of months due to augmentation-amplification effects of insulin.

### EXAMPLE 3

Use the insulin preparation as described above: Apply one drop in each eye conjunctival sac. Apply pressure at nasolacrimal sac at the medial canthus-nasal junction (FIG. 4) to prevent the leaking of the insulin drop to the nasal mucosa with subsequent development of systemic complication of hypoglycemia. This maneuver is optional and precautionary in hypoglycemic individuals. Then apply one drop of aqueous cyclosporin water soluble eye preparation as formulated in the invention U.S. Patent Application Publication Number: US 2010/0016219 Al. Insulin can enhance the uptake of water soluble cyclosporin than oil soluble preparations; and augment-amplify the effects of the cyclosporins on the structures involved in development of dry eye syndrome. Due to use of insulin, the effect of cyclosporins lasts longer and helps to restore the lacrimal secretions and do not have to wait for 6 months to achieve the effect as seen in oily Cyclosporin preparations. Insulin not only has effect on dry eye syndrome; on its own right, it also augments the effect of other therapeutic, pharmaceutical, biochemical and biological agents or compounds in the dry eye syndrome treatment preparations.

### EXAMPLE 4

Non Sjogren's syndrome KCS is a common suffering in postmenopausal women, pregnant women, women who are taking oral contraceptives, or in menopausal women on hormone replacement therapy with estrogens. The common denominator here is a decrease in androgens, either from reduced ovarian function in the postmenopausal female or from increased levels of the sex hormone binding globulin in pregnancy and birth control pill use. Androgens are believed to be trophic for the lacrimal and meibomian glands. They also exert potent anti-inflammatory activity through the production of transforming growth factor beta (TGF-beta), suppressing lymphocytic infiltration. The testosterone containing eye drops can be prepared used after pretreatment with insulin. The ophthalmic drops can be prepared using testosterone (androgen), DHEA—a mild androgen, cyclosporin and insulin can be used to treat the Sjogren's syndrome, KCS and other dry eyes syndrome. Our preliminary studies indicate, that is easy to prepare these ophthalmic eye preparations to treat dry eye syndrome with different etiological factors.

### EXAMPLE 5

It has been shown that in meibomian gland dysfunction (Meibomian gland dysfunction (MGD)), due to a deficiency in androgens results in loss of the lipid layer, particularly triglycerides, cholesterol, monounsaturated essential fatty acids (e.g., oleic acid), and polar lipids (e.g., phosphatidylethanolamine, sphingomyelin) contributed by the meibomian gland (glands of Zeis) secretions. This loss of polar lipids (present at the mucin-aqueous-tear interface) exacerbates the tear loss evaporation. This also leads to the decrease in unsaturated fatty acids raises the melting point of meibum, leading to thicker, more viscous secretions. This obstructs the ductules resulting in obstruct to flow of secretions.

Interestingly, the Patients on anti androgenic therapy for prostate disease also have increased viscosity of meibum, decreased tear break-up time, and increased tear film debris, all indicative of a deficient or abnormal tear film. Application of testosterone locally on the eye lid followed after insulin application can help the uptake of testosterone by the meibomian glands, rejuvenating these glands to start secreting and produce physiologically healthy oily secretions to relieve dry eye syndrome.

### EXAMPLE 6

Signs of a dry eye are variable depending on the state of the disease i.e. mild, moderate, severe or very severe form. The following are the noticeable signs: Bulbar conjunctival vascular dilation, Decreased tear meniscus, Irregular corneal surface, Decreased tear break-up time, Punctuate epithelial keratopathy, Corneal filaments, Increased debris in the tear film, Conjunctival pleating, Superficial punctuate keratitis, with positive fluorescein staining, Mucous discharge and even Corneal ulcers in severe cases. These cases are treated with insulin drops two to three times a day followed by cyclosporin ophthalmic preparations. In severe cases, insulin drops, testosterone and cyclosporin drops are alternated. If there is severe infection, systemic administration along with local instillation of antibiotics and/or antiviral with insulin as describe is warranted.

### EXAMPLE 7

Sjögren's syndrome (also known as “Mikulicz disease” and “Sicca syndrome”) is labeled as an autoimmune disorder in which immune cells attack and destroy the salivary and lacrimal exocrine glands. It affects 4 million people, second only to rheumatic disease. 90% of patients are woman above the age of 40, although any age group of men and woman can be affected. It can be primary or developing years after rheumatoid arthritis, systemic lupus erythematosus, scleroderma, primary biliary cirrhosis etc named as Secondary Sjögren's syndrome. There is no cure for Sjogren's syndrome dry eye syndrome and the treatment is palliative. Artificial tears, goggles to increase local humidity or punctual plugs inserted to help retain tears are of temporary relief. Our invention of insulin as single agent or other therapeutic, pharmaceutical, biochemical and biological agents or compounds are already available through pharmaceutical companies becomes more effective. First use insulin ophthalmic drops followed by the following selected anti Sjogren's syndrome therapeutic agents. Our choice is, after insulin eye drops, instill cyclosporin, or methotrexate eye drops followed by insulin drops after 3-5 minutes.

### EXAMPLE 8

Treatment of Sjogren's syndrome can be achieved also by using our inventive method of using insulin. Instill insulin ophthalmic drops. Wait 3-5 minutes. Then administer methotrexate eye drops. Methotrexate is cheap, easily available chemo therapeutic agents used in autoimmune diseases and cancers. It is known that the in the presence of insulin, the methotrexate therapeutic agents effect is increased up to 10,000 fold is (Oliver Alabaster' et al. Metabolic Modification by Insulin Enhances Methotrexate Cytotoxicity in MCF-7 Human Breast Cancer Cells, Eur J Cancer Clinic; 1981, Vol 17, pp 1223-1228). Methotrexate is water soluble. The dose given orally for Sjogren's syndrome and rheumatoid arthritis is 5 Mg per 70 kilo weight person. That amounts to about 70 micrograms per kilogram body weight.

Because of insulin augmentation-amplification effects, we prepare and use 1 to 5 microgram per ml. depending on the severity of Sjogren's syndrome. In mild cases we use only 1-2 mcg of methotrexate with 0.5 IU of rapid acting insulin. In severe cases use up to 5 micrograms of methotrexate combination ophthalmic drops. Another therapeutic agent that can be used is cyclophosphamide. We have used this therapeutic agent to treat autoimmune disease with insulin very successfully for treating scleroderma and systemic lupus erythematosus. Cyclophosphamide is a synthetic anti neoplastic drug chemically related to the nitrogen mustards, soluble in water, saline, or ethanol. It is used 1-5 mg kg body weight in the treatment of autoimmune diseases. We have used the therapeutic agents successfully to treat scleroderma. Dilute the agent in distilled water to a concentration of 5-10 μg per ml. This should be used as ophthalmic drops after insulin instillation or combined with insulin as described above.

### EXAMPLE 9

The symptoms associated with dry eye are frequently exacerbated using contact lenses. The contact lens intolerance is caused in part, or total, by the condition of dry eye and its symptoms. The rate of evaporation from the eye is accelerated by the contact lens surface. For many subjects, contact lens intolerance is not overcome by topical application of tear substitutes; hence there is a need for improved compositions and processes for treatment for improving tolerance to ocular prostheses. Our method of using insulin mixed therapeutic, pharmaceutical, biochemical and biological agents or compounds in its formulation can help this sometimes unbearable condition.

### EXAMPLE 10

Antibodies are proteins that are generated by the immune system white blood cells. They circulate in the blood and attach to foreign proteins called antigens in order to destroy or neutralize them to help rid the systemic infection or eliminate foreign proteins harmful to the body cells. Monoclonal antibodies are laboratory produced substances that can locate and bind to specific molecules such as tumor necrosis factor (TNF), a protein involved in causing the inflammation and damage of rheumatoid arthritis and autoimmune diseases like Sjogren's syndrome. Remicade™, Etanercept, Embresl™, and Humira™ are such TNF anti TNF agents in the market to treat autoimmune bodies.

Etanercept is a drug that used to treat autoimmune diseases by interfering with the tumor necrosis factor (TNF, a part of the immune system) by acting as a TNF inhibitor. It is given 25-50 mg. Humira administered by injection is produced from fully human proteins. The newest monoclonal protein to be approved for the treatment of rheumatoid arthritis is Rituxan. Infliximab (Remicade), is a chimeric mouse/human monoclinal antibody given by intravenous infusion and works by binding to tumor necrosis factor alpha (TNFα).

Several new monoclonal antibodies are in the development stage to treat rheumatoid arthritis and other conditions. Sjogren's syndrome like rheumatoid arthritis's, being a autoimmune disease, Multiple monoclonal antibodies are currently under investigation for the treatment of this condition producing dry eye syndrome (Meijer J M, Pijpe J, Bootsma H, Vissink A, Kallenberg C G (June 2007). “The future of biologic agents in the treatment of Sjögren's syndrome”. Clin Rev Allergy Immunol 32 (3): 292-7). All TNF inhibitors are immunosuppressant's. For example, the Etanercept (Embrel) is given in the dose of 25 mg bi weekly in Rheumatoid arthritis in a 70 Kg person which amounts to 350 μg per KG body weight. We advise using no more than 200 μg per ml, which results in 10 μg per drop instilled. Instill this final solution with insulin as described above to treat Sjogren's syndrome. Use the insulin and MAB preparations once or twice a day. First dose to be administered at bed time. The dose of MAB given is minuscule; to take any contraindications such as tuberculosis into account while using these biological therapeutic agents with our invention insulin.

### EXAMPLE 11

The hyaluronic acid (HA) is produced by fermenting the bacterial strain Bacillus subtilis. It is the world's first pure HA that is 100% free of animal-derived raw materials and organic-solvent remnants. Hyaluronic acid is an novel visco-enhancer for use in topical eye care formulations which is also filterable and heat stabile with pH (0.1% solution) 6.0-7.5 which is ideally suited to treat dry eye syndrome and Sjogren's syndrome. The HA can be a key ingredient for topical ophthalmic formulations since it is a natural compound that is biocompatible, non-immunogenic, and biodegradable.

It is one of the most hygroscopic molecules found in nature; hydrated hyaluronic acid can contain up to 1,000-fold more water than its own weight. These exceptional water retention properties result in enhanced hydration of the corneal surface in dry eye syndrome. Moreover, applications of ophthalmic formulations containing HA reduce tear elimination and enhance pre corneal tear film stability, which is a useful property against dry eye syndrome. The muco-adhesivity of Hyaluronic acid provides effective coating and long lasting protection of the cornea and conjunctival sac due to its extended stay, water retention quality and accommodation times on the ocular surface. When topically instilled on the eye with insulin, HA promotes physiological wound healing by stimulating corneal epithelial migration and proliferation of keratocytes as well as reducing the healing time of corneal epithelium in advanced severe cases of dry eye syndrome. HA as the viscosity-enhancing agent of choice, decreases the drainage rate of ophthalmic solutions.

In eye drops designed for drug delivery, a highly viscous HA solution prolongs the contact time of the drug with the cornea, resulting in improved bioavailability of the drug when other therapeutic, pharmaceutical, biochemical and biological agents or compounds are mixed. Thus, Hyaluronic acid-containing formulations can be heat sterilized under standard conditions without compromising the final attributes of the formulations. Our invention of using insulin before and after the application HA with or without Cyclosporins or combining with insulin in the final formulation can effectively prevent, curtail and cure the dry eye syndrome associated with or without Sjogren's syndrome and Meibomian gland dysfunction (MGD).

### EXAMPLE 12

Mitoxantrone (Novantrone) is a chemotherapeutic drug that works by suppressing the immune system. It is used to slow the worsening of neurologic disability and reduce the relapse rate in patients with clinically worsening forms of relapsing-remitting and secondary progressive MS. Mitoxantrone, a DNA-reactive agent that intercalates into deoxyribonucleic acid (DNA) through hydrogen bonding, causes cross links and strand breaks. Mitoxantrone also interferes with ribonucleic acid (RNA) and is a potent inhibitor of topoisomerase II, an enzyme responsible for uncoiling and repairing damaged DNA. It can be prepared in doses of 100 μg/ml. by premixing with insulin also. These drops can be effective in autoimmune related dry eye syndrome.

### EXAMPLE 13

Corticosteroids are one of the most commonly used for the treatment for multiple sclerosis; autoimmune diseases, allergic conditions, insect bites, and septic shock and many other conditions. They are given to reduce the inflammation that spikes during a relapse of multiple sclerosis. Examples include oral prednisone and intravenous methylprednisolone. Lotemax, an ophthalmic corticosteroid, targets inflammation with a unique, site-active mechanism of action. Structural modifications associated with an ester ophthalmic steroid make Lotemax highly lipid soluble, enhancing penetration into cells and enabling Lotemax to exert anti-inflammatory activity within the eye. Pre treating with insulin or combining with insulin can enhance the further uptake of this corticosteroids and relive Sjogren's syndrome and dry eye syndrome. Lotemax is indicated for the treatment of steroid responsive inflammatory conditions associated with the palpebral and bulbar conjunctiva, cornea, and anterior segment of the globe. Adding our invention insulin as delineated above can only help its therapeutic effectiveness for which it is used.

### EXAMPLE 14

Homeopathic drops such as active Comparator Drug (euphrasia based homeopathic therapy) and cyclosporin ophthalmic solution are being devised to treat the dry eye syndrome. It is still under study and the results are still being evaluated. Use of micrograms of insulin drops into eye before the use of any of the above described therapeutic agents can act as homeopathic dose and enhance their therapeutic activity.

### EXAMPLE 15

Methylsulfonylmethane (MSM), is an organosulfur compound with the formula (CH₃)₂SO2, a metabolite of DMSO. It is also known by several other names: DMSO2, MSM, Methylsulfonylmethane, methyl sulfone, and dimethyl sulfone. It is marketed as dietary supplement and also used for osteoarthritis, allergic rhinitis, interstitial cystitis, and throat spray for snoring. Stanley W. Jacob, M.D., of the Oregon Health and Science University, reports using MSM to treat over 18,000 patients with a assortment of ailments (MSM the Definitive Guide: The Nutritional Breakthrough for Arthritis, Allergies and More. Freedom Press. 2003. ISBN 9781893910225. http://www.amazon.com/MSM-Definitive-Guide-Nutritional-Breakthrough/dp/1893910229). It is soluble in water, and a good solvent like DMSO. We have used aqueous solutions of MSM, filtered and sterilized and mixed with insulin and used as eye drops to treat dry eye syndrome during chemotherapy, allergic conditions of the eyes and contact lens wearers with good results.

### EXAMPLE 16

There are many therapeutic agents under development. Diquafosol (INS365 Ophthalmic) developed by the Inspire Pharmaceuticals is one of them. It works in an independent mechanism via P2Y2 receptors to stimulate fluid secretion from non-lacrimal gland tissue, and that would be expected to help patients who might not be able to benefit from Restasis. Another ophthalmic drop under development is Rebamipide ophthalmic suspension (Novartis), a medication that causes mucus secretion, is currently used orally in Japan as ulcer therapy to stimulate mucus production by the lining of the stomach. The topical preparation being investigated for dry eye is in Phase III clinical trials in the United States. It is evaluated for clinical study at three concentrations: 0.5%, 1% and 2%. Results showed that with use of the 2% suspension fluorescein corneal staining, as well as subjective parameters such as gritty/sandy sensation and burning/pain, were decreased from baseline (Donshik P, et al. IOVS 2005; 46: ARVO E-Abstract #2037). They should be used with our invention of Insulin eye drops. Insulin can be used before instilling these eye drops or used in combination, in a preparation where the insulin is added to the formulations of Diquafosol and Rebamipide to make them more effective with very low concentration.

### EXAMPLE 17

There is high incidence of dry eye syndrome (keratoconjunctivitis sicca) in the postmenopausal women with symptoms ranging from mild foreign body, pain and even visual loss due to ocular surface abnormalities. Decades back the use of conjugated estrogens to treat KCS (Bohigian, G. Handbook of External Diseases of the Eye (Alcon, Inc.) 1980, p. 79). U.S. Pat. No. 5,041,434; U.S. Pat. No. Re. 34,578; and U.S. Pat. No. 6,096,733 describe the use of estrogens. The latter patent disclosed very small doses of 17-β-estradiol Compounded with polysorbate 80 (USP), povidone (USP) (K-30 type), hydroxyethylcellulose (USP), sodium chloride (USP), disodium EDTA (USP), benzalkonium chloride (USP), dilute HCL for pH adjustment and purified water (USP) qs.

As described in our invention pre treating the affected eyes with insulin or adding to the above preparation of estradiol eye drop can enhance the local therapeutic effect by insulin mediated augmentation-amplification effects and provide the needed relief much faster without systemic effect. Administration of systemic estradiol is not needed which can have adverse effect on the other systems.

### EXAMPLE 18

The symptoms of eye allergy are mild to moderate and can be severe during early spring and beginning of fall. Self treatment such as avoiding allergy triggers, irrigating eyes with saline (salt water) and placing packs and cold water compresses on your eyes may not be effective in severe cases and the medical treatment is needed to relieve them. Severe allergic conjunctivitis that isn't helped by other treatments may benefit from specific allergen immunotherapy (desensitization) which is usually effective. Most commonly used and prescribed medications are: levocabastine (brand name Livostin); antihistamines (antolozine) together with a medicine that constricts blood vessels (naphazoline, phenylephrine); sodium cromoglycate (4%); non-steroidal anti-inflammatory (NSAID) eye drops; and steroids (hydrocortisone, Dexamethasone, prednisolone). Eye drops containing vasoconstrictors or cortisone, must not be used long term without the supervision of an ophthalmologist.

### EXAMPLE 19

The present invention of topical ophthalmic drops use of insulin relates to compositions to include vasoconstrictor components for treating ocular hyperemia, without causing eye irritation and/or a dual treatment of hyperemia and dry eye. Reddening or inflammation of the superficial tissues of the eye is a very common affliction due to various allergic reactions, foreign body irritation in the eye, trauma, systemic bleeding disorders, result of malignant hypertension, use of anticoagulants or eye fatigue. The superficial conjunctival redness frequently referred to as hyperemia or ocular hyperemia, can be the result of ciliary body congestion, dilation of the deep straight vessels of the episclera, and/or dilation of the superficial vessels of the conjunctiva.

The most common treatment includes the administration of eye drops which contain emollients and ingredients to eliminate the redness associated with the condition. Many of the commercially available vasoconstrictor eye drops include EDTA and other preservatives which may produce discomfort when used. Topical vasoconstrictor (causes narrowing of the blood vessels) eye medications can cause ‘rebound’ effects, encouraging overuse as the eye gets red again as the effect wears off. It is claimed that the Sodium cromoglycate eye drops rarely cause side effects. Steroid drops are effective in relieving symptoms quickly, but may be linked with cataract formation, glaucoma and bacterial and viral infections of the cornea and conjunctiva if they're used long term. Along with insulin, various vasoconstrictors can be use such as: alpha-1-adrenergic agonists include tetrahydrozoline, ephedrine, naphazoline, phenylephrine, tetrahydrozoline, vasoxyl, oxymetazoline, or any and/or salts and mixtures thereof. Due to trophic activity of Insulin, the rebound effect of the vasoconstrictor ophthalmic is reduced or eliminated.

Demulcent (a substance that soothes irritated or inflamed skin or mucous membranes such as Lanoline and glycerin) component 0.1% to about 5 (w/V). when added can be effective in lubricating an eye such as polyanionic components, hydroxyethylcellulose, hydroxypropylmethyl cellulose, methylcellulose, dextran, gelatin, glycerin, polyethylene glycols, for example: polyethylene glycol 300, polyethylene glycol 400 and the like, polysorbates, propylene glycol, polyvinyl alcohol, polyvinyl pyrrolidone and the like and mixtures thereof. See U.S. Pat. Nos. 4,421,748 and 5,474,979, the disclosure of each of which is incorporated in its entirety herein by reference.

### EXAMPLE 20

Studies has shown subjectively the patients felt better when DHEA ophthalmic drops were use compared to the artificial tears or testosterone (Connor C G, and Fender J. Comparison of Androgenic Supplemented Artificial Tears. (Invest Ophthalmol Vis Sci 2002; 43: E-Abstract 66; Schaumberg D A, Sullivan D A, Dana M R. Epidemiology of dry eye syndrome. Adv Exper Med Biol 2002; 506: 989-998. Schaumberg D A, Sullivan D A, Buring J E, Dana M R. Prevalence of dry eye syndrome among US women. Am J Ophth 2003; 136:318-326). This study supports the previous studies by Notion and Sullivan that addition of androgenic hormones to artificial tears benefit dry eye patients. DHEA also known as dehydroepiandrosterone is a steroid hormone produced by the adrenal glands and converted to other hormones like estrogen and testosterone. It is a steroid hormone produced naturally by the adrenal glands that has 5% of the androgenic activity of testosterone.

Also if the testosterone or DHEA eye drops are used to treat Sjogren's syndrome and dry eye syndromes, first use insulin drops before the application of this eye preparation to the eye then use the ophthalmic preparation. Even better, prepare these hormonal eye drops in combination with insulin and then use as ophthalmic drop to treat this conditions.

### EXAMPLE 21

Oral intake of Vitamin C, GLA, omega 3, and vitamin B6 can be of help who suffer from dry eye syndrome and contact lens users. These supplements will increase the tear production and help the condition of dry eye syndrome. Insulin drops should be used 30 minutes after taking these supplements to enhance their uptake by the lid glandular system.

### EXAMPLE 22

A method of topically instilling insulin drops to a person or animals conjunctiva and its sac to treat dry eye syndrome with or without Sjogren's syndrome, and Meibomian gland dysfunction (MGD), with administration of insulin and then enhance their uptake and therapeutic activity by using into afflicted structures in the eye can be enhanced combined with uptake facilitators such electroporation, iontophoresis, sonophoresis, vibroacoustic, vibration and other physical (heat, magnetic force, radio frequency, microwave, laser lights etc) methods with other appropriate therapeutic, biological and pharmacological anti-glaucoma and retinal protectors agents combined with insulin therapy as described. These methods can be used as prophylaxis, to diagnose, prevent and to treat the above conditions.

### EXAMPLE 23

Another drug available to treat KCS is a organo sulfur compound, anethole dithiolethione (ADT—trade name Sialor) with hardly any side effects. It stimulates the secretion of saliva, in patients with autoimmune exocrinopathy (Sjogren's syndrome). Sialor alleviates the symptoms of xerostomia and xeroophthalmia. We have used ADT 25 mg orally and in nanograms concentration in liquid ophthalmic eye drops with successes in these conditions especially those on chemotherapy, menopausal women and chronic smokers with dry mouth and dry eyes conditions.

This can be one of the important non toxic oral and eye drops for the treatment of dry eye syndrome (Ben-Mandi M H, Gozin A, Driss F, Andrieu V, Christen M O, Pasquier C. Anethole dithiolethione regulates oxidant-induced tyrosine kinase activation in endothelial cells. Antioxid Redox Signal. 2000 Winter;2(4):789-99). Studies by Han et al show that ADT is more bioavailable lipid-based formulations, as sub-micro emulsion (SME) and oil solution prepared using short (SCT), medium (MCT) and long (LCT) chain triglycerides respectively. (Han S F, Yao T T, Zhang X X, Gan L, Zhu C, Yu H Z, Gan Y. Int J Pharm. Lipid-based formulations to enhance oral bioavailability of the poorly water-soluble drug anetholtrithione: effects of lipid composition and formulation. 2009 Sep. 8; 379(1):18-24. Epub 2009 Jun. 7.). The emulsion or water soluble compound of ADT ophthalmic drops can be used after insulin drops. Insulin can be combined with the formulation to instill to the eye with one dispenser. The ADT is non toxic and can be very efficacious in treating dry eye syndrome prophylactically and by the contact lens users.

### EXAMPLE 24

Many times, the dry eye syndrome is produced by the infection of the eye lids resulting what is commonly known as “Pink Eye” It can be infectious or non infectious. Pink eye, or conjunctivitis, is redness and inflammation of the conjunctival covering the whites of the eyes and the membranes on the inner part of the eyelids without any vision changes. These membranes react to a wide range of bacteria, viruses, allergy-provoking agents, irritants, and toxic agents, as well as to underlying diseases within the body.

The Viral and bacterial forms of conjunctivitis are common in childhood, but they can occur in any age. Bacterial pink eye is caused by Staphylococci and Streptococci, among others treated with antibiotic eye drops. Pink eye, due to infection with Chlamydia, is an uncommon form of bacterial pink eye in the U.S., but it is very common in Africa and Middle Eastern countries treated with erythromycin, and tetracycline. Viral pink eye do not respond to antibiotics, and will pass away in a week. The bacterial infections related to pink eye are treated with antibiotic/antiviral eye drops combined with insulin as described above.

### EXAMPLE 25

Use of Chelation Therapeutic Agents with Insulin

It is a known fact that the many gland ducts of the meibomian glands and other secretory glands and secretory apparatuses of the glands gets deposits of fat, calcium, protenacious and dead cell complexes with advancing age. It is highly likely; they do have many metallic and organic deposits such as lipoprotenacious material, iron and calcium in them due to death of cells and protenacious deposits. Chelation therapy with Ethylenediaminetetraacetic acid (EDTA), Methylsulfonylmethane (MSM), Alagebrium, and Deferoxamine (also known as desferrioxamine B, desferoxamine B, DFO-B, DFOA, DFB or desferal) will clear the clogged secretory acini and duct system of the eye glandular system. They remove any metal, calcium, and other metallic as well as protenacious deposits which affect their physiological role in the proper secretory functioning of the lacrimal and lid glands.

Ethylenediaminetetraacetic acid (EDTA) unclogs blood vessels; controls free radical damage due to lipid peroxidation by serving as a powerful antioxidant; increases tissue flexibility by uncoupling age-related cross-linkages that are responsible for loss of cellular function; removes lead, cadmium, aluminum, and other metals, restoring enzyme systems to their proper functions; enhances the integrity of cellular and mitochondrial membranes; reduces the tendency of platelets to cause coagulation too readily which can clog the glandular system; unclogs the clogged draining vascular system, increases tissue flexibility by uncoupling age-related cross-linkages (age related glycation) that are responsible proper function of the glands. The use of EDTA along with insulin as described in our invention can slow down, arrest or reverse the changes in these secretory glands and establish increased secretions and flow of the secretions.

Deferoxamine is a chelating agent used to remove excess iron from the body. By removing excess iron, it reduces the damage done to various organs and tissues, such as the liver, CNS, eyes, trabecular meshwork and retina. The damage we see in retina can be due to excessive iron form the choroid and retinal blood vessels leaking excessive iron. The role of iron (metallobiology) in neurodegenerative disorders has long been implicated, with particular attention given to iron as it is one of the most important redox metals, which have been largely linked to senile toxicity and neurodegenerative disorders such as Alzheimer's and Parkinson's diseases and aging patients (Stankiewicz J M, Brass S D (2009) Role of iron in neurotoxicity: a cause for concern in the elderly? Curr Opin Clin Nutr Metab Care 12:22-9).

The redox switching capability of iron from ferrous to ferric state, and vice versa, makes it one of the most dangerous catalytic elements responsible for the secretory and neurodegenerative process. Iron generates free radicals and reactive oxygen species in the aged tissue as evidenced by higher heme oxygenase-I, which contributes to increased susceptibility to oxidative stress in older people (Hirose W, Ikematsu K, Tsuda R (2003) Age-associated increase in heme oxygenase-1 and ferritin immunoreactivity in the autopsied brain. Leg Med 5(Suppl. 1):360-6).

The nerve tissue that stimulates the secretions is not spared by the neurodegenerative process by iron. Biochemical events surrounding iron-mediated catalytic events, which give rise to oxidative stress and free radical generation, can be described as known Fenton reaction as indicated below.

Fe3+.O²→KO2−Fe2⁺+O₂ (Step I);

Fe²++H₂O₂−Fe³⁺+OH−+KOH (Step II)

Combining Step I and II: .O2⁻+H₂O₂→.HO⁻+O2

The role of iron in the neurodegenerative process can be best described in three distinct phases: accumulation, invasion, and catalytic activity. A recent study also shows that it speeds healing of nerve damage (and minimizes the extent of recent nerve trauma), glandular and their nerve supply damage in dry eye syndrome can be reduced or curtailed by iron Chelation. Deferoxamine may modulate expression and release of inflammatory mediators seen in autoimmune diseases of the dry eye syndrome such as Sjogren's syndrome as indicated in Fenton reaction by specific cell types thus reduce or stop the damage by our invention. Using it with insulin can enhance its activity and restore tear production from the glandular system of the lids.

Methylsulfonylmethane (MSM) is a supplement form of sulfur that is found in our living tissues. MSM supports healthy connective tissues like tendons, ligaments, and muscle and glandular function. MSM makes cell walls permeable, allowing water and nutrients to freely flow into cells and allowing wastes and toxins to properly flow out. MSM is an anti-oxidant that helps to clean the blood stream and flush toxins trapped in our cells. It is also a foreign protein and free radical scavenger which is needed to maintain the glandular system functions which need to produce the lacrimal fluids. The body uses MSM along with Vitamin C to create new, healthy cells, and MSM provides the flexible bond between the cells. Using MSM with insulin as eye drops can alleviate the dry eye syndrome.

We prepare the following eye drops containing: 1. EDTA, 2. Deferoxamine, 3. MSM, with added preservatives, anti bacterial and DMSO combined with insulin in proper concentrations. Any one of the chelating agent or combination of them can be used to formulate the eye drops. These eye drops are used before or after insulin drops a prophylactic and therapeutic agents for DES, as well as to treat glaucoma, cataract and lower the IOP as well.

### EXAMPLE 26

Alagebrium (known as ALT-711) is the first drug to be clinically tested for the purpose of breaking the crosslink's caused by advanced glycation end products (AGEs), thereby reversing one of the main mechanisms of aging and seen in diabetics at an early age. The drying seen in the diabetics and aged can be related to AGEs due to carbohydrates binding to proteins including structural proteins, lipids, and DNA. This process can impair the normal function of organs (including tear producing glands) that depend on flexibility and proper nutrition supply for normal functioning. AGEs cross links leads to loss of function of tissues, induce oxidative stress, in which reactive molecules provoke the underlying component of inflammation. Hence the Alagebrium eye drops in combination with Insulin can prevent AGEs formation, facilitate their removal and reverse the disease state affecting the tear production and relieve the DES.

### EXAMPLE 27

Insulin composition for diagnosing the health and disease of the eye such as in diagnosing the dry eye syndrome, said composition comprising a solution containing a combination of dyes (sodium fluorescein, rose Bengal, and lissamine green) where one of said dyes is a pharmaceutically acceptable dye capable of staining lid wiper, cornea and scleral defective epithelium cells (FIGS. 1-3) and another of said dyes is a pharmaceutically acceptable dye capable of staining lid wiper degenerated epithelium cells. First use insulin drops, wait for 3-5-10 minutes, and then use the selected eye drops. Insulin will enhance the rapid uptake of the dyes and helps to diagnose the wipers afflictions of the eye lid involved in dry eye syndrome. This method relates for diagnosing a deficiency in the anatomy and performance of the upper eyelid; a recognition of the impact of this deficiency during blinking on problems such as dry eye, contact lens intolerance and ocular discomfort in general; and the use of this diagnostic method to provide a treatment modality to alleviate such problems.

### EXAMPLE 28

There are conditions of the DES with other afflictions can lead to pathology of the eye and eye lids with or without ulcerations of the cornea, sclera and the eye lids affecting the growth of epithelial cells, fibroblasts, and blood vessels. Growth factors are naturally occurring or synthesized substance capable of stimulating cellular growth, proliferation and cellular differentiation. Typically it is a protein or a steroid hormone. They are important for regulating a variety of cellular processes and typically act as signaling molecules between cells. Examples are cytokines and hormones that bind to specific receptors on the surface of their target cells. Use of insulin with growth factors such as Epidermal growth factor (EGF), vascular growth factors, Insulin-like growth factor (IGF), Nerve growth factor (NGF), platelet-derived growth factor, fibroblast growth factor, blood vessel related growth and anti angiogenesis factors; and other growth factors can be used with insulin as eye drops to treat eye advanced eye diseases. Insulin, IGF-1, and Somatomedin are also growth factors that can be used in many DES related diseases needing the growth and differentiation of the cornea, conjunctiva and other glandular system which contribute to the tears. Hyperemia of the eye and excessive blood vessel growth of the eyes can be disturbing in many diseases of the eye. Such conditions can be treated with anti angiogenesis factors such as bevacizumab (Avastin) with or without vasoconstrictors and/or anti allergic therapeutic agents with insulin as described in this invention.

Numerous modifications; alternative arrangements of steps explained and examples given herein may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form function and manner of procedure, assembly and use may be made. While the preferred embodiment of the present invention has been described, it should be understood that various changes, adaptations and modifications may be made thereto. It should be understood, therefore, that the invention is not limited to details of the illustrated invention. This method can also be used to diagnose corneal ulcers; any pathological changes in the cornea and conjunctiva of the eye.

While the preferred embodiment of the present invention has been described, it should be understood that various changes, adaptations and modifications may be made thereto. It should be understood, therefore, that the invention is not limited to details of the illustrated invention examples.

What is claimed is:

1. A method of treating dry eye syndrome comprising the step of administering a therapeutically effective amount of insulin to an afflicted eye conjunctival sac.

2. The method of treating dry eye syndrome according to claim 1 further comprising the step of applying a therapeutic agent to said afflicted eye.

3. A method of treating dry eye syndrome, comprising the step of administering a therapeutically effective amount of IGF-1 to an afflicted eye.

4. The method of treating dry eye syndrome according to claim 3 further comprising the step of applying a therapeutic agent to said afflicted eye.

5. The method of treating dry eye syndrome according to claim 2 wherein said therapeutic agent is a pharmaceutical agent.

6. The method of treating dry eye syndrome according to claim 2 wherein said therapeutic agent is a biochemical agent.

7. The method of treating dry eye syndrome according to claim 2 wherein said known therapeutic agent is selected from a group consisting of cyclosporins in aqueous base and oily base.

8. The method of treating dry eye syndrome due to autoimmune disease according to claim 2 wherein said known therapeutic agent is a biological agents containing Monoclonal Antibodies; IFN-α and agents targeting TNF-α and B cells (anti-CD20, anti-CD22) and prostaglandins, growth factors, and anti angiogenesis growth factors such as bevacizumab (Avastin).

9. The method of treating dry eye syndrome according to claim 2 wherein said known therapeutic agent consisting of administration of testosterones; estrogens; Hydroxychloroquine (Plaquenil) and azathioprine (Imuran).

10. The method of treating dry eye syndrome according to claim 2 wherein said known therapeutic agents are ophthalmic preparations of Anetholdithiolthione (ADT, 5-[p-methoxyphenyl]-3H-1,2-dithiol-3-thione); pilocarpine (Salagen), and cevimeline (Evoxac), Alagebrium and anethole dithiolethione (ADT).

11. The method of treating dry eye syndrome according to claim 2 wherein said known therapeutic agent is a combination of two agents selected from the group comprising cyclosporins and testosterone.

12. The method of treating dry eye syndrome according to claim 2 wherein said known therapeutic agent is extract of turmeric (Curcumin), and dehydroepiandrosterone (DHEA).

13. The method of treating dry eye syndrome according to claim 2 wherein therapeutic agent are methotrexate, Cyclophosphamide and Mitoxantrone (Novantrone).

14. The method of treating dry eye syndrome according to claim 2 wherein said known therapeutic agent is hyaluronic acid.

15. The method of treating dry eye syndrome according to claim 2 wherein said therapeutic agent comprising of Diquafosol (INS365 Ophthalmic) and Rebamipide.

16. The method of treating dry eye syndrome according to claim 2 wherein said known therapeutic agent are chelating agents Methylsulfonylmethane (MSM), Ethylenediaminetetraacetic acid (EDTA), Alagebrium and Deferoxamine.

17. The method of treating dry eye syndrome according to claim 2 further comprising the step of using an uptake facilitator to further enhance the therapeutic effect selected from the group comprising electroporation, iontophoresis, sonophoresis, vibroacoustic, vibration, physical heat, magnetic field, radio frequency field, microwave and laser light.

18. The method of treating dry eye syndrome according to claim 2 wherein said known therapeutic agents are Vitamin C, Gamma linolenic acid (GLA is an omega 6 fatty acid), omega 3 fatty acid, vitamin B6, vitamin E drops; phospholipids, liposome-based; retinoids; glycerin, propylene glycol, glutathione, and calcium ion ophthalmic drops compositions.

19. The method of treating dry eye syndrome according to claim 2 wherein said known therapeutic agents are an antibiotic, and antiviral.

20. The method of treating dry eye syndrome due to allergic condition according to claim 2 wherein said known therapeutic agents are levocabastine (Livostin); antihistamines (antolozine, Pheniramine maleate), vasoconstrictors (naphazoline hydrochloride, phenylephrine); Naphazoline hydrochloride, sodium cromoglycate; Naphcon A, non-steroidal anti-inflammatory drugs (NSAID); Ketorolac trimethamine; and corticosteroids (hydrocortisone, Dexamethasone, prednisolone).
